SEQUENCE LISTING (8 sequences)

SEQ ID NO: 1 - protein, 171 aa, Drosophila melanogaster
DSTPVGKLRQMPPFKMIYPSYGNVAGSHDGMLGGGCLPYGKNTNDKQPWLKDYLQQWKSSDRFRSRAMPHIKSYTRFNLEDQSVYWFVLTSANLSKAAWGCFNKNSNIQPCLRIANYEAGVLFLPRFVTGEDTFPLGNNRDGVPAFPLPYDVPLTPYAPDDKPFLMDYLQG

SEQ ID NO: 2 - protein, 319 aa, Mus musculus
HQKTQGIWLSPLYPRIDQGSHTAGESSTRFKADLTSYLTAYNAPPLQEWIDIIQEHDLSETNVYLIGSTPGRFQGSHRDNWGHFRLRKLLQAHAPSTPKGECWPIVGQFSSIGSLGPDESKWLCSEFKDSLLALREEGRPPGKSAVPLHLIYPSVENVRTSLEGYPAGGSLPYSIQTAEKQRWLHSYFHKWSAETSGRSNAMPHIKTYMRPSPDFSKLAWFLVTSANLSKAAWGALEKNGTQLMIRSYELGVLFLPSAFGLDTFKVKQKFFSSSCEPTASFPVPYDLPPELYGSKDRPWIWNIPYVKAPDTHGNMWVPS

SEQ ID NO: 3 - protein, 264 aa, Homo sapiens
  VARIANT 111, 112, 113, 114, 115, 116, 117, 118, 119, 120, 121,122, 123, 124: Xaa = Any Amino Acid
ETNVYLIGSTPGRFQGSQKDNWGHFRLKKLLKDHASSMPNAESWPVVGQFSSVGSLGASESKWLCSEFKESMLTLGKESKTPGKSSVPLYLIYPSVENVRTSLEGYPAGGXXXXXXXXXXXXXXKQNWLHSYFHKWSAETSGRSNAMPHIKTYMRPSPDFSKIAWFLVTSANLSKAAWGALEKNGTQLMIRSYELGVLFLPSAFGLDSFKVKQKFFAGSQEPMATFPVPYDLPPELYGSKDRPWIWNIPYVKAPDTHGNMWVPS

SEQ ID NO: 4 - protein, 343 aa, Homo sapiens
LGWCLSSSDDELQPEMPQKQAEKVVIKKEKDISAPNDGTAQRTENHGAPACHRLKEEEDEYETSGEGQDIWDMLDKGNPFQFYLTRVSGVKPKYNSGALHIKDILSPLFGTLVSSAQFNYCFDVDWLVKQYPPEFRKKPILLVHGDKREAKAHLHAQAKPYENISLCQAKLDIAFGTHHTKMMLLLYEEGLRVVIHTSNLIHADWHQKTQGIWLSPLYPRIADGTHKSGESPTHFKADLISYLMAYNAPSLKEWIDVIHKHDLSETNVYLIGSTPGRFQGSQKDNWGHFRLKKLLKDHASSMPNAESWPVVGQFSSVGSLGADESKWLCSEFKESMLTLGKES

SEQ ID NO: 5 - protein, 451 aa, Caenorhabditis elegans
MKRTIQETPGPSSTTVPPPKKLNSQRNGSNLEPGSIYFTPIGGISVPRQESESSRSLDEILADIRPINSLHFSFMLDFEFLIGSYPPSLREYPITLVVGAPDAPDLLKCTKNQKLVTVVGASLPIPFGTHHTKMSILEDEDGRFHVIVSTANLVPDDWEFKTQQFYYNFGVKIASGTVPRSDFQDDLLEYLSMYRNQLDTWKQLLQKVDFSQISDRLIFSTPGYHTDPPTQRPGHPRLFRILSEKFPFDASYEHTERCTFVAQCSSIGSLGSAPINWFRGQFLQSLEGANPSPKQKPAKMYLVFPSVEDVRTSCQGYAGGCSVPYRNSVHARQKWLQGNMCKWRSNAKRRTNAVPHCKTYVKYDKKVAIWQLLTSANLSKAAWGEVSFNKSKNVEQLMIRSWEMGVLITDPSRFNIPFDYPLVPYSATDEPFVTDKKHEKPDILGCIWTPP

SEQ ID NO: 6 - protein, 544 aa, Saccharomyces cerevisiae
MSRETNFNGTKRKRSDVAEKVAQRWKSVRYSAEMENMAPVNSNNDSDDCVIVSESKIIDLTNQEQDLSERIETNDTAKGAVFKLMKSDFYEREDFMGEVEDMITLKDIFGTETLKRSILFSFQYELDFLLRQFHQNVENITIVGQKGTIMPIEARAMDATLAVILKKVKLIEITMPPFASHHTKLIINFYDNGECKIFLPSNNFTSMETNLPQQVCWCSPLLKIGKEGLPVPFKRSLIEYLNSYHLKDIDELITKSVEEVNFAPLSELEFVYSTPSKFQSSGLLSFYNKLEKLSAGTSASDTAKHYLCQTSSIGTSLSRARDENLWTHLMIPLFTGIMSPPAKDTAGRKKAEILPTNSLINEYSQRKIKPYIIFPTEQEFVTSPLKWSSSGWFHFQYLQKKSYYEMLRNKFKVFYKQDPAMVTRRRGTTPAHSKFYMHCATNSAGPCDASQVFKELEWCLYTSANLSQTAWGTVSRKPRNYEAGVLYHSRRLANTRKVTCRTFTRDRRGCAGNPTHVAVPFTLPVIPYDLAEDECFCLARHEND

SEQ ID NO: 7 - DNA, 2344 bp, Homo sapiens
  CDS (251)...(2074)
gttggttctgtgcgcctcagagttggagcacacagctgtattaaaaaggcaaatcgaaggccgggcgcggtgactcacgcctgtcatcctagcactttgggaggccgaggcggctgaatcacttgaggttaggagtttgagatcagcccgggcaacatggtgaaaccccgtctctacaaaaatagaaaaattagccgagcgtgatggtggatgcctgtaatcctagctcctcgggaggctaaggagtataatgtctcaggaaggcgattatgggaggtggaccatatctagtagtgatgaaagtgaggaagaaaagccaaaaccagacaagccatctacctcttctcttctctgtgccaggcaaggagcagcaaatgagcccaggtacacctgttccgaggcccagaaagctgcacacaagaggaaaatatcacctgtgaaattcagcaatacagattcagttttacctcccaaaaggcagaaaagcggttcccaggaggacctcggctggtgtctgtccagcagtgatgatgagctgcaaccagaaatgccgcagaagcaggctgagaaagtggtgatcaaaaaggagaaagacatctctgctcccaatgacggcactgcccaaagaactgaaaatcatggcgctcccgcctgccacaggctcaaagaggaggaagacgagtatgagacatcaggggagggccaggacatttgggacatgctggataaagggaaccccttccagttttacctcactagagtctctggagttaagccaaagtataactctggagccctccacatcaaggatattttatctcctttatttgggacgcttgtttcttcagctcagtttaactactgctttgacgtggactggctcgtaaaacagtatccaccagagttcaggaagaagccaatcctgcttgtgcatggtgataagcgagaggctaaggctcacctccatgcccaggccaagccttacgagaacatctctctctgccaggcaaagttggatattgcgtttggaacacaccacacgaaaatgatgctgctgctctatgaagaaggcctccgggttgtcatacacacctccaacctcatccatgctgactggcaccagaaaactcaaggaatatggttgagccccttatacccacgaattgctgatggaacccacaaatctggagagtcgccaacacattttaaagctgatctcatcagttacttgatggcttataatgccccttctctcaaggagtggatagatgtcattcacaagcacgatctctctgaaacaaatgtttatcttattggttcaaccccaggacgctttcaaggaagtcaaaaagataattggggacattttagacttaagaagcttctgaaagaccatgcctcatccatgcctaacgcagagtcctggcctgtcgtaggtcagttttcaagcgttggctccttgggagccgatgaatcaaagtggttatgttctgagtttaaagagagcatgctgacactggggaaggaaagcaagactccaggaaaaagctctgttcctctttacttgatctatccttctgtggaaaatgtgcggaccagtttagaaggatatcctgctgggggctctcttccctatagcatccagacagctgaaaaacagaattggctgcattcctattttcacaaatggtcagctgagacttctggccgcagcaatgccatgccacatattaagacatatatgaggccttctccagacttcagtaaaattgcttggttccttgtcacaagcgcaaatctgtccaaggctgcctggggagcattggagaagaatggcacccagctgatgatccgctcctacgagctcggggtccttttcctcccttcagcatttggtctagacagtttcaaagtgaaacagaagttcttcgctggcagccaggagccaatggccacctttcctgtgccatatgatttgcctccagaactgtatggaagtaaagatcggccatggatatggaacattccttatgtcaaagcaccggatacgcatgggaacatgtgggtgccctcctgagaatcttgaggcactgtgaaatttaagtgtaagacattgagccacaaacatggaatctcttctttgtactggatgtccacttcccttaaagtcttatttgcacccttacaaaatctttccaaaggtcactcttatgaatggatgttggttatacttttaatggacattaacattcctaataaagtattagtttcttaaaaaaaaaaaaaaaaaaaaaaaaaaaaaaaaaaaaaaaaaaaaaaaaaaaaaaaaaaaaaaaaaaaaaaa

SEQ ID NO: 8 - protein, 608 aa, Homo sapiens
MSQEGDYGRWTISSSDESEEEKPKPDKPSTSSLLCARQGAANEPRYTCSEAQKAAHKRKISPVKFSNTDSVLPPKRQKSGSQEDLGWCLSSSDDELQPEMPQKQAEKVVIKKEKDISAPNDGTAQRTENHGAPACHRLKEEEDEYETSGEGQDIWDMLDKGNPFQFYLTRVSGVKPKYNSGALHIKDILSPLFGTLVSSAQFNYCFDVDWLVKQYPPEFRKKPILLVHGDKREAKAHLHAQAKPYENISLCQAKLDIAFGTHHTKMMLLLYEEGLRVVIHTSNLIHADWHQKTQGIWLSPLYPRIADGTHKSGESPTHFKADLISYLMAYNAPSLKEWIDVIHKHDLSETNVYLIGSTPGRFQGSQKDNWGHFRLKKLLKDHASSMPNAESWPVVGQFSSVGSLGADESKWLCSEFKESMLTLGKESKTPGKSSVPLYLIYPSVENVRTSLEGYPAGGSLPYSIQTAEKQNWLHSYFHKWSAETSGRSNAMPHIKTYMRPSPDFSKIAWFLVTSANLSKAAWGALEKNGTQLMIRSYELGVLFLPSAFGLDSFKVKQKFFAGSQEPMATFPVPYDLPPELYGSKDRPWIWNIPYVKAPDTHGNMWVPS

(19) United States
(12) Patent Application Publication
(10) Pub. No.: US 2003/0105053 A1
(43) Pub. Date: Jun. 5, 2003

(54) Therapeutic compositions and screening methods relating to 3' -tyrosyl-DNA-phosphodiesterase

(76) Inventors: Alex B. Burgin JR., Bainbridge Island, WA (US); Marc Rideout, Bainbridge Island, WA (US)

Correspondence Address: CATHRYN CAMPBELL, CAMPBELL & FLORES LLP, 7th Floor, 4370 La Jolla Village Drive, San Diego, CA, 92122, US

(21) Appl. No.: 10/216,052

(22) Filed: Aug. 8, 2002

Related U.S. Application Data
(60) Provisional application No. 60/311,058, filed on Aug. 8, 2001.

(51) Int. Cl. A61K 48/00; A61K 31/7076; A61K 31/7072; A61K 31/4745; C07H 21/04

(52) U.S. Cl. 514/44; 514/45; 514/283; 536/23.1

(57) ABSTRACT

The invention provides a compound that inhibits the activity of 3′-tyrosyl-DNA phophodiesterase (TDP). In one embodiment, the compound is a polynucleotide-3′-bridging phosphoramidate. The invention also provides a method of inhibiting TDP activity. In one embodiment, the method involves contacting the enzyme with a polynucleotide-3′-bridging phosphoramidate, polynucleotide-3′-alkyl phosphonate, polynucleotide-3′-alkyl phosphotriester, polynucleotide-bridging-alkyl phosphonate, nucleotide-3′-bridging phosphoramidate, nucleotide-3′-alkyl phosphonate, nucleotide-3′-alkyl phosphotriester, or nucleotide-bridging-alkyl phosphonate. The invention further provides a method of decreasing cellular proliferation. The method involves contacting a TDP-containing cell with an effective amount of a TDP inhibiting compound sufficient to inhibit TDP activity in said cell. In addition, the invention provides a method of screening for compounds that modulate the activity of TDP.

This application is based on, and claims the benefit of, U.S. Provisional Application No. 60/311,058, filed Aug. 8, 2001, which is incorporated herein by reference.

### SUMMARY OF THE INVENTION

The invention provides a compound that inhibits the activity of 3′-tyrosyl-DNA phophodiesterase (TDP). In one embodiment, the compound is a polynucleotide-3′-bridging phosphoramidate. The invention also provides a method of inhibiting TDP activity. In one embodiment, the method involves contacting the enzyme with a polynucleotide-3′-bridging phosphoramidate, polynucleotide-3′-alkyl phosphonate, polynucleotide-3′-alkyl phosphotriester, polynucleotide-bridging-alkyl phosphonate, nucleotide-3′-bridging phosphoramidate, nucleotide-3′-alkyl phosphonate, nucleotide-3′-alkyl phosphotriester, or nucleotide-bridging-alkyl phosphonate.

The invention further provides a method of decreasing cellular proliferation. The method involves contacting a TDP-containing cell with an effective amount of a TDP inhibiting compound sufficient to inhibit TDP activity in said cell.

In addition, the invention provides a method of screening for compounds that modulate the activity of TDP. The method consists of contacting TDP, or a fragment or modification thereof having TDP activity, with a compound under conditions that allow TDP activity, determining an amount of TDP activity, and identifying a compound that modulates TDP activity. The compounds so identified are potentially useful therapeutic agents for decreasing cell proliferation.

### BACKGROUND OF THE INVENTION

Cancer is currently the second leading cause of mortality in the United States. However, it is estimated that by the year 2000 cancer will surpass heart disease and become the leading cause of death in the United States. Cancerous tumors result when a cell escapes from its normal growth regulatory mechanisms and proliferates in an uncontrolled fashion. Tumor cells can metastasize to secondary sites if treatment of the primary tumor is either not complete or not initiated before substantial progression of the disease. Early diagnosis and effective treatment of tumors is therefore essential for survival.

Continuous developments over the past quarter century have resulted in substantial improvements in the ability of a physician to diagnose a cancer in a patient. Unfortunately, methods for treating cancer have not kept pace with those for diagnosing the disease. Thus, while the death rate from various cancers has decreased due to the ability of a physician to detect the disease at an earlier stage, the ability to treat patients presenting more advanced disease has progressed only minimally.

A hurdle to advances in treating cancer is the relative lack of agents that can selectively target the cancer, while sparing normal tissue. For example, radiation therapy and surgery, which generally are localized treatments, can cause substantial damage to normal tissue in the treatment field, resulting in scarring and, in severe cases, loss of function of the normal tissue. Chemotherapy, which generally is administered systemically, can cause substantial damage to organs such as bone marrow, mucosae, skin and the small intestine, which undergo rapid cell turnover and continuous cell division. As a result, undesirable side effects, for example, nausea, hair loss and reduced blood cell counts, occur as a result of systemically treating a cancer patient with chemotherapeutic agents. Such undesirable side effects often limit the amount of a treatment that can be administered.

A related hurdle prohibiting significant advances in cancer treatment is the relative lack of cancer specific targets. For example, treatments have been attempted where cytotoxic agents have been directed to tumor cell surface markers. However, such approaches similarly result in pleiotropic side effects and damage to normal tissue because of the lack of availability or specificity of tumor cell markers. Due to such shortcomings in treatment, cancer remains a leading cause of patient morbidity and death.

Thus, there exists a need for improved methods of treating cancer and other proliferative pathological conditions. The present invention satisfies this need and provides related advantages as well.

### BRIEF DESCRIPTION OF THE DRAWINGS

FIG. 1 shows PAGE analysis of purified 3′-tyrosyl-DNA-phosphodiesterase.

FIG. 2 shows HPLC analysis of 3′-derivatized polynucleic acids.

FIG. 3 shows 3′-tyrosyl-DNA-phosphodiesterase activity.

FIG. 4 shows kinetic analysis of 3′-tyrosyl-DNA-phosphodiesterase. FIG. 4A shows the velocity of cleavage of 3′-(4′-nitro-phenyl)-oligonucleotides plotted as a function of substrate concentration. FIG. 4B shows an Eadie-Hofstee plot of the data presented in FIG. 4A.

FIG. 5 shows an exemplary synthesis scheme for oligonucleotides containing a 3′-phospho-phenyl group in which the phenyl moiety is post-synthetically derivatized.

FIG. 6 shows a comparison of two TDP reaction mechanisms.

FIG. 7 shows a comparison of mechanism based inhibitors.

FIG. 8 shows an exemplary synthetic combinatorial library.

FIG. 9 shows a scheme for deconvoluting a combinatorial library.

FIG. 10 shows the chemical structures of a 3′-tyrosyl-DNA-phosphodiesterase substrate and the corresponding product, the formation of which can be monitored spectrophotometrically.

FIG. 11 shows the chemical structures of a 3′-tyrosyl-DNA-phosphodiesterase substrate and the corresponding product, the formation of which can be monitored in a fluorescence-based assay.

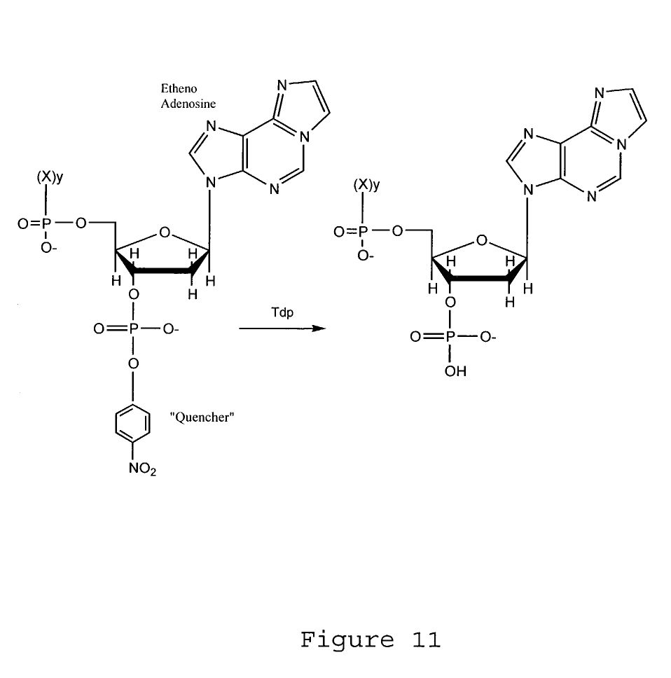

FIG. 12 shows an alignment of the amino acid sequences of TDP polypeptide sequences from drosphila melanogasler (SEQ ID NO:1), mouse (SEQ ID NO:2), human (SEQ ID NOS:3 and 4), C.elegans (SEQ ID NO:5) and S.cerevisiae (SEQ ID NO:6)(Pouliot, et al. Science, 286:552-555 (1999)).

### DETAILED DESCRIPTION OF THE INVENTION

This invention is directed to inhibitors of 3′-tyrosyl-DNA phosphodiesterase (TDP) activity. TDP is an enzyme that cleaves the chemical bond between a topoisomerase and a DNA molecule to which topoisomerase is bound. Topoisomerases are cellular enzymes that function by breaking the DNA backbone, allowing DNA to undergo topological change, and then resealing the break. During this process, topoisomerases form a covalent bond with the DNA prior to the resealing step. Under circumstances in which the resealing step fails, topoisomerase remains covalently bound to the DNA. Such topoisomerase-DNA complexes disrupt normal cellular replication, leading to cell death. For example, DNA mismatches, nicked DNA, camptothecin-like drug induced- and topoisomerase-induced mutation have been shown to cause covalent complexes to accumulate in vitro (Yeh et al. J. Biol. Chem., 269:15498-15405 (1994), Lanza et al. J. Biol. Chem 271:6989-6986 (1996)). TDP is a repair enzyme that prevents the accumulation of these faulty covalent topoisomerase-DNA complexes that normally occur during the life time of a cell. Inhibiting TDP activity thus promotes cell death by allowing the accumulation of topoisomerase-DNA complexes. Therefore, inhibitors of TDP provide a means of destroying unwanted cells, such as pathologically aberrant cells associated with proliferative diseases, infectious disease and other disorders of excessive or unwanted cell proliferation.

In one embodiment, the invention is directed to compounds that inhibit TDP activity. Such compounds include nonhydrolyzable analogs, such as derivatized polynucleotides, which act as competitive inhibitors of TDP activity. The compounds of the invention are useful for inhibiting TDP contained in a variety of samples, as well as in cells, tissues and organs of an animal, including a human.

As used herein, the term “polynucleotide-3′-bridging phosphoramidate” is intended to mean a polynucleotide that contains at least one “3′-bridging phosphoramidate moiety.” As used herein, the term “3′-bridging phosphoramidate moiety” is intended to mean a nucleotide having at least one phosphate group linked by a phosphodiester bond at the 3′ position of the sugar ring, the phosphate group having a phosphate oxygen substituted by nitrogen. The nitrogen can be bound to a chemical group, in particular a chemical group that would result in the formation of a poor leaving group in a reaction in which the nitrogen atom is attacked by a nucleophile. The nitrogen atom forms a bridge or link between the bound phosphorus atom and a bound chemical group.

A “polynucleotide-3′-bridging phosphoramidate” is a compound having the following structure:

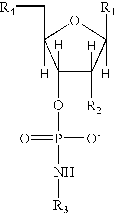

wherein, X is a nucleotide; y is a positive integer; R1 is any nucleotide base; R2 is H, OH, halo, amino, alkyl or azido; and R3 is phenyl, aryl, substituted phenyl or substituted aryl.

A “nucleotide-3′-bridging phosphoramidate” is a compound having the following structure:

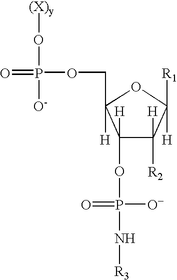

wherein, X is a nucleotide; y is a positive integer; R1 is any nucleotide base; R2 is H, OH, halo, amino, alkyl or azido; and R3 is phenyl, aryl, substituted phenyl or substituted aryl; and R4 is a hydrogen atom, hydroxyl, azido, halo, amino or O-alkyl.

As used herein, the term “polynucleotide-3′ phosphorothioate” is intended to mean a polynucleotide that contains at least one 3′-phosphorothioate moiety. As used herein, the term “3′-phosphorothioate moiety” is intended to mean a nucleotide having at least one phosphate group linked by a phosphodiester bond at the 3′ position of the sugar ring, the phosphate group having a phosphate oxygen substituted by sulfur.

A “polynucleotide-3′ phosphorothioate” is a compound having the following structure:

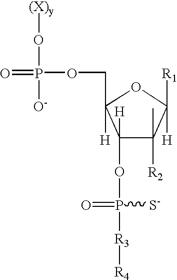

wherein, X is a nucleotide; y is a positive integer; R1 is a nucleotide base; R2 is a hydrogen atom, hydroxyl, halo amino, alkyl or azido; R3 is oxygen; and R4 is a halo, alkyl, substituted alkyl, phenyl or substituted phenyl.

An exemplary polynucleotide-3′ phosphorothioate contains a phenyl moiety at the R₄ position, such as:

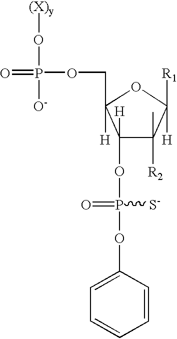

A “nucleotide-3′ phosphorothioate” is a compound having the following structure:

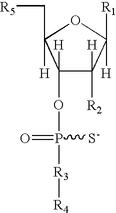

wherein, X is a nucleotide; y is a positive integer; R1 is a nucleotide base; R2 is a hydrogen atom, hydroxyl, halo, amino, alkyl or azido; R3 is oxygen; R4 is a halo, alkyl, substituted alkyl, phenyl or substituted phenyl; and R5 is a hydrogen atom, hydroxyl, azido, amino, halo, or O-alkyl.

As used herein, the term “polynucleotide-3′-alkyl phosphonate” is intended to mean a polynucleotide that contains at least one 3′-alkyl phosphonate moiety. As used herein, the term “3′-alkyl phosphonate moiety” is intended to mean a nucleotide having at least one phosphate group linked by a phosphodiester bond at the 3′ position of the sugar ring, the phosphate group having a phosphate oxygen substituted by an alkyl group, such as a methyl or ethyl group and a phosphate oxygen substituted by another moiety, R3.

A “polynucleotide-3′ alkyl phosphonate” is a compound having the following structure:

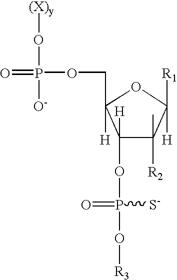

wherein, X is a nucleotide; y is a positive integer; R1 is a nucleotide base; R2 is a hydrogen atom, hydroxyl, halo amino, alkyl or azido; R3 is a halo, alkyl, substituted alkyl, phenyl or substituted phenyl; and R4 is alkyl.

An exemplary polynucleotide-3′ alkyl phosphonate contains a phenyl moiety at the R3 position, such as:

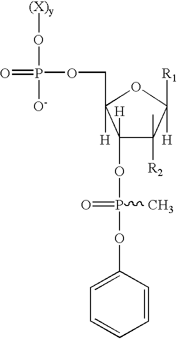

A nucleotide-3′ alkyl phosphonate is a compound having the following structure:

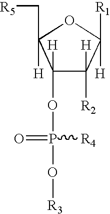

wherein, X is a nucleotide; y is a positive integer; R1 is a nucleotide base; R2 is a hydrogen atom, hydroxyl, halo amino, alkyl or azido; R3 is halo, alkyl, substituted alkyl, phenyl or substituted phenyl; R4 is alkyl; and R5 is a hydrogen atom, hydroxyl, azido, halo, amino, or O-alkyl.

As used herein, the term “polynucleotide-3′-alkyl phosphotriester” is intended to mean a polynucleotide that contains at least one 3′-alkyl phosphotriester moiety. As used herein, the term “3′-alkyl phosphotriester moiety” is intended to mean a nucleotide having at least one phosphate group linked by a phosphodiester bond at the 3′ position of the sugar ring, the phosphate group having a phosphate oxygen substituted by a phosphoester group.

A “polynucleotide-3′-alkyl phosphotriester” has the following structure:

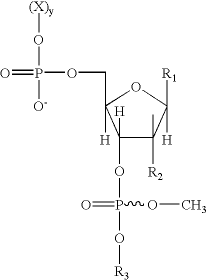

wherein, X is a nucleotide; y is a positive integer; R1 is a nucleotide base; R2 is a hydrogen atom, hydroxyl, halo amino, alkyl or azido; and R3 is a halo, alkyl, substituted alkyl, phenyl or substituted phenyl.

A “nucleotide-3′-alkyl phosphotriester” has the following structure:

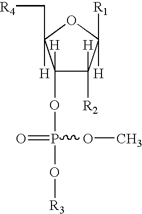

wherein, X is a nucleotide; y is a positive integer; R1 is a nucleotide base; R2 is a hydrogen atom, hydroxyl, halo amino, alkyl or azido; R3 is a halo, alkyl, substituted alkyl, phenyl or substituted phenyl; and R4 is a hydrogen atom, hydroxyl, azido, halo, amino, or O-alkyl.

As used herein, the term “polynucleotide-bridging-alkyl phosphonate” is intended to mean a polynucleotide that contains at least one bridging-alkyl phosphonate moiety. As used herein, the term “bridging-alkyl phosphonate moiety” is intended to mean a nucleotide having at least one phosphate group linked by a phosphodiester bond at the 3′ position of the sugar ring, the phosphate group having a phosphate oxygen substituted by a substituted alkyl group.

A “polynucleotide-bridging-alkyl phosphonate” has the following structure:

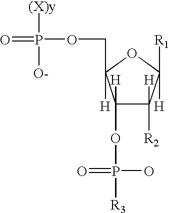

wherein, X is a nucleotide; y is a positive integer; R1 is a nucleotide base; R2 is a hydrogen atom, hydroxyl, halo amino, alkyl or azido; R3 is substituted alkyl.

An exemplary “polynucleotide-bridging-alkyl phosphonate” is:

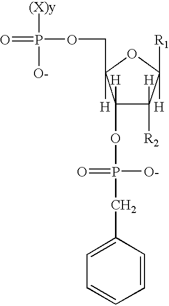

A “nucleotide-bridging-alkyl phosphonate” has the following structure:

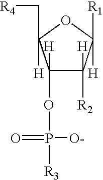

wherein, X is a nucleotide; y is a positive integer; R1 is a nucleotide base; R2 is a hydrogen atom, hydroxyl, halo amino, alkyl or azido; R3 is substituted alkyl; and R4 is a hydrogen atom, hydroxyl, azido, halo, amino, or O-alkyl.

As used herein, the term “polynucleotide” is intended to mean a chain of two or more nucleotide 5′-monophosphate residues linked through one or more phosphodiester bonds. A nucleotide of a polynucleotide can contain a variety of glycose moieties, such as, for example, D-ribose and D-2-deoxyribose, as well as modified glycose moieties such as cytarabine. Therefore, a polynucleotide encompasses ribonucleic acid (RNA) or deoxyribonucleic acid (DNA), a hybrid of RNA and DNA, as well as RNA and DNA molecules containing nucleotides which have modified glycose moieties.

A nucleotide, including a nucleotide contained in a polynucleotide, can contain any nucleic acid base, including both naturally occurring and modified bases. Examples of naturally occurring bases include guanine, adenine, thymine, cytosine and uridine. Examples of modified bases include bases that are detectable by a variety of analytical methods, for example, fluorescent bases and bases with useful absorbance characteristics. Exemplary modified bases include 4-thio-uridine, pseudouridine, 2′-deoxy-uridine, 5-fluoro-uridine, 5-bromo-uridine, 5-iodo-uridine, 2′-amino-uridine, 2′-fluoro-uridine, 2′-fluoro-cytidine, 2′-amio-butyryl-pyrene-uridine, 5-fluoro-cytidine, ribo-thymidine, 5-methyl-cytidine, inosine, purine ribonucleoside, 2-aminopurine, 2,6-diaminopurine, N3-methyl uridine and ribavirin. A variety of other structures can be incorporated into a synthetic base. Particularly useful structures include those that render a molecule detectable, favorably alter an activity of the molecule or function as purification tags. For example, a base can contain 3′ and 5′ modifications such as 3′-puromycin, 3′-inverted deoxy thymidine, 3′-thioate linkage, 5′-biotin, a fluorescent moiety such as 5′-fluorescein, 5′-Cy3, 5′-Cy5, 5′-tetrachloro-fluorescein, and other moieties, with and without atomic spacers.

A nucleotide or polynucleotide can be naturally occurring or synthetically produced. For example, a polynucleotide can be isolated from an organism or synthesized using various methods, such as automated methods well known in the art. A naturally occurring polynucleotide can be, for example, an RNA such as an mRNA, a DNA such as a cDNA or genomic DNA, and can represent the sense strand, the anti-sense strand, or both.

A polynucleotide can be a single stranded, duplex or branched polynucleotide. A duplex polynucleotide is a polynucleotide having two strands associated together by hydrogen bonding. A strand of a duplex polynucleotide can contain one or more mismatched, absent or additional nucleotides that do not associate with the cognate nucleotide in the partner strand, so long as the duplex remains associated. A duplex polynucleotide includes polynucleotides of both synthetic and natural origin. Thus, a duplex polynucleotide can be, for example, cDNA, genomic DNA, RNA, mRNA, synthetic DNA, including, for example, annealed complementary oligonucleotides or polynucleotides. A polynucleotide of natural origin can be derived from any eukaryotic, prokaryotic or viral source. Duplex DNA can have blunt ends, 3′ terminal end overhangs and 5′ terminal end overhangs. Single stranded, duplex or branched DNA further can contain a tag or moiety, such as a tag useful for detection or purification.

The term “alkyl” denotes such radicals as methyl, ethyl, n-propyl, isopropyl, n-butyl, sec-butyl, tert-butyl, amyl, tert-amyl, hexyl and the like. A preferred alkyl group is methyl.

The term substituted alkyl groups is intended to mean alkyl groups, such as C1 to C6 and C7 to C12 alkyl groups that are substituted by one or more halogen, hydroxy, protected hydroxy, oxo, protected oxo, cyclohexyl, naphthyl, amino, protected amino, (monosubstituted)amino, protected (monosubstituted)amino, (disubstituted)amino, guanidino, heterocyclic ring, substituted heterocyclic ring, imidazolyl, indolyl, pyrrolidinyl, C1 to C7 alkoxy, C1 to C7 acyl, C1 to C7 acyloxy, nitro, C1 to C7 alkyl ester, carboxy, protected carboxy, carbamoyl, carboxamide, protected carboxamide, N-(C1 to C6 alkyl)carboxamide, protected N-(C1 to C6 alkyl)carboxamide, N,N-di(C1 to C6 alkyl)carboxamide, cyano, methylsulfonylamino, thio, C1 to C4 alkylthio or C1 to C4 alkyl sulfonyl groups. The substituted alkyl groups can be substituted once or more with the same or with different substituents.

Examples of the above substituted alkyl groups include the chloromethyl, bromomethyl, iodomethyl, trifluoromethyl, 6-hydroxyhexyl, 2,4-dichloro(n-butyl), 2-aminopropyl, chloroethyl, bromoethyl, fluoroethyl, iodoethyl, chloropropyl, bromopropyl, fluoropropyl, iodopropyl and the like.

The term “substituted phenyl” specifies a phenyl group substituted with one or more, and preferably one or two, moieties chosen from the groups consisting of halogen, hydroxy, protected hydroxy, cyano, nitro, C1 to C6 alkyl, C1 to C7 alkoxy, C1 to C7 acyl, C1 to C7 acyloxy, carboxy, protected carboxy, carboxymethyl, protected carboxymethyl, hydroxymethyl, protected hydroxymethyl, amino, protected amino, (monosubstituted)amino, protected (monosubstituted)amino, (disubstituted)amino, carboxamide, protected carboxamide, N-(C1 to C6 alkyl)carboxamide, protected N-(C1 to C6 alkyl)carboxamide, N, N-di(C1 to C6 alkyl)carboxamide, trifluoromethyl, N-((C1 to C6 alkyl)sulfonyl)amino, N-(phenylsulfonyl)amino or phenyl, substituted or unsubstituted, such that, for example, a biphenyl results.

Examples of the term “substituted phenyl” include a mono- or di(halo)phenyl group such as 2, 3 or 4-chlorophenyl, 2,6-dichlorophenyl, 2,5-dichlorophenyl, 3,4-dichlorophenyl, 2, 3 or 4-bromophenyl, 3,4-dibromophenyl, 3-chloro-4-fluorophenyl, 2, 3 or 4-fluorophenyl and the like; a mono or di(hydroxy)phenyl group such as 2, 3 or 4-hydroxyphenyl, 2,4-dihydroxyphenyl, the protected-hydroxy derivatives thereof and the like; a nitrophenyl group such as 2, 3 or 4-nitrophenyl; a cyanophenyl group, for example, 2, 3 or 4-cyanophenyl; a mono- or di(alkyl)phenyl group such as 2, 3 or 4-methylphenyl, 2,4-dimethylphenyl, 2, 3 or 4-(iso-propyl)phenyl, 2, 3 or 4-ethylphenyl, 2, 3 or 4-(n-propyl)phenyl and the like; a mono or di(alkoxyl)phenyl group, for example, 2,6-dimethoxyphenyl, 2, 3 or 4-methoxyphenyl, 2, 3 or 4-ethoxyphenyl, 2, 3 or 4-(isopropoxy)phenyl, 2, 3 or 4-(t-butoxy)phenyl, 3-ethoxy-4-methoxyphenyl and the like; 2, 3 or 4-trifluoromethylphenyl; a mono- or dicarboxyphenyl or (protected carboxy)phenyl group such as 2, 3 or 4-carboxyphenyl or 2,4-di(protected carboxy)phenyl; a mono-or di(hydroxymethyl)phenyl or (protected hydroxymethyl)phenyl such as 2, 3, or 4-(protected hydroxymethyl)phenyl or 3,4-di(hydroxymethyl)phenyl; a mono- or di(aminomethyl)phenyl or (protected aminomethyl)phenyl such as 2, 3 or 4-(aminomethyl)phenyl or 2,4-(protected aminomethyl)phenyl; or a mono- or di(N-(methylsulfonylamino))phenyl such as 2, 3 or 4-(N-(methylsulfonylamino))phenyl. Also, the term “substituted phenyl” represents disubstituted phenyl groups wherein the substituents are different, for example, 3-methyl-4-hydroxyphenyl, 3-chloro-4-hydroxyphenyl, 2-methoxy-4-bromophenyl, 4-ethyl-2-hydroxyphenyl, 3-hydroxy-4-nitrophenyl, 2-hydroxy 4-chlorophenyl and the like.

The terms “halo” and “halogen” refer to fluoro, chloro, bromo or iodo groups.

As used herein, the term “3′-tyrosyl-DNA-phosphodiesterase” or “TDP,” refers to a class of enzymes that hydrolyze a 3′-tyrosyl phosphodiester, such as a 3′-tyrosyl phosphodiester bond between a topoisomerase and the 3′ end of a polynucleotide. An exemplary TDP is yeast TDP. Nucleotide and amino acid sequences of yeast TDP are available in the GenBank database as accession number Z36092.1. EST sequences corresponding to TDP genes of other organisms, including human, mouse, Drosophila melanogaster and Caenorhabditis elegans are available in the GenBank database. For example, ESTs corresponding to human TDP include AA477148, AA489121 and AI480141. An exemplary human TDP nucleotide sequence (SEQ ID NO:7), which encodes amino acid sequence SEQ ID NO:8 is available at GenBank accession number NM_{—}018319.

TDP functions to cleave a covalent 3′-tyrosyl phosphodiester bond between a topoisomerase and a polynucleotide. Therefore, binding to a topoisomerase-polynucleotide complex is a “TDP activity.” Another “TDP activity” is cleavage of a 3′-tyrosyl phosphodiester bond, such as a 3′-tyrosyl bond between topoisomerase and a polynucleotide. As used herein, the term “TDP enzymatic activity” is intended to mean both binding of TDP to a topoisomerase-polynucleotide complex and cleavage of a 3′-tyrosyl bond between topoisomerase and a polynucleotide.

The term TDP encompasses native TDP from a variety of species, and also encompasses polypeptides containing minor modifications of a native TDP sequence, and fragments of a full-length native TDP, so long as the modified polypeptide or fragment retains one or more biological activities of a native TDP, such as the abilities to bind to topoisomerase-DNA complexes, and cleave a 3′-tyrosyl phosphodiester bond. A modification of a TDP can include additions, deletions, or substitutions of amino acids, so long as a biological activity of a native TDP is retained. For example, a modification can serve to alter the stability or activity the polypeptide, or to facilitate its purification.

A modified TDP can contain amino acid analogs, derivatives and mimetics. Such modifications and functional equivalents of amino acids are well known to those skilled in the art. Amino acid analogs include modified forms of naturally and non-naturally occurring amino acids. Such modifications can include, for example, substitution or replacement of chemical groups and moieties on the amino acid or by derivitization of the amino acid. Amino acid mimetics include, for example, organic structures which exhibit functionally similar properties such as charge and charge spacing characteristic of the reference amino acid. For example, an organic structure which mimics arginine would have a positive charge moiety located in similar molecular space and having the same degree of mobility as the ε-amino group of the side chain of the naturally occurring amino acid. Those skilled in the art know or can determine what structures constitute functionally equivalent amino acid analogs and amino acid mimetics.

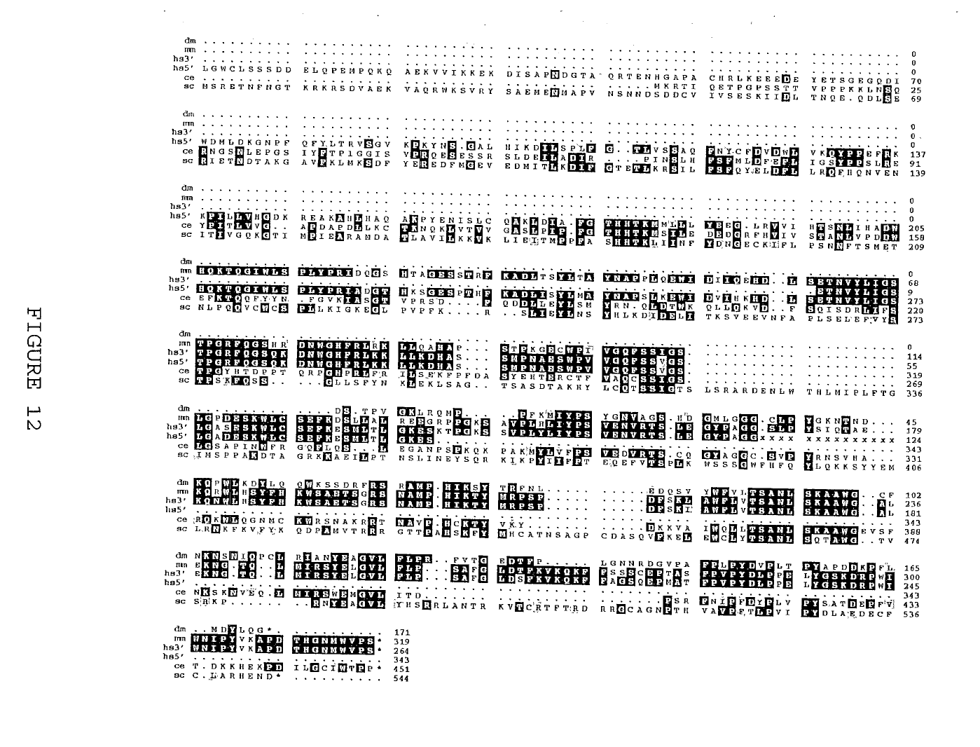

A TDP can be modified, for example, to increase polypeptide stability, alter a TDP activity, facilitate detection or purification, or render the enzyme better suited for a particular application, such as by altering substrate specificity. Computer programs known in the art can be used to determine which amino acid residues of a TDP can be modified as described above without abolishing a TDP activity (see, for example, Eroshkin et al., Comput. Appl. Biosci. 9:491-497 (1993)). In addition, structural and sequence information can be used to determine the amino acid residues important for TDP activity. For example, a TDP crystal structure (Davies et al. Structure 19(2):237-248 (2002)) and comparisons of TDP amino acid sequences, such as that shown in FIG. 12 can provide guidance in determining amino acid residues that can be altered without abolishing TDP activity.

A “fragment” of a TDP is intended to mean a portion of a TDP that retains a portion of the activity of a native TDP, and can retain at least about the same activity as a native TDP. A fragment of a TDP can contain a modification. It is understood that the activity of a TDP can be measured by the methods disclosed herein.

The term “TDP” includes native or recombinantly expressed TDP expressed in cells or cell lysates, and includes chemically synthesized TDP. A TDP can contain an exogenous amino acid sequence, such as, for example, a tag that facilitates purification or identification. Exemplary tags include histidine tags, glutathione-S transferase tags, FLAG tags and myc tags. Other chemical tags such as biotin and fluorescent or radioactive tags can be present on a TDP polypeptide or nucleic acid molecule.

As used herein, the term “TDP modulating” when used in reference to a compound is intended to mean that the compound alters the amount or rate of a TDP activity, such as a binding interaction between TDP and a topoisomerase-polynucleotide complex or 3′-tyrosyl phosphodiesterase activity, compared to a reference level of TDP activity. A TDP modulating compound can thus be, for example, a compound that selectively binds TDP, a compound that modulates an interaction between TDP and a substrate, such as a topoisomerase-polynucleotide complex or derivatized polynucleotide, a compound that modulates 3′-tyrosyl phophodiesterase activity, or a compound with more than one of these effects. A TDP modulating compound can increase or decrease the amount or rate of a TDP activity. A TDP modulating compound can act directly or indirectly. A TDP modulating compound that acts directly can, for example, bind to a TDP and alter substrate affinity, substrate specificity, or susceptibility to proteolysis. A TDP modulating compound that acts indirectly can, for example, bind to a molecule that regulates the expression level or activity of TDP, thereby altering the amount or rate of TDP activity. Therefore, TDP modulating compounds include a “TDP inhibiting” compound. As used herein, the term “TDP inhibiting” when used in reference to a compound is intended to mean a compound that decreases, directly or indirectly, the amount or rate of TDP activity. A TDP inhibiting compound can, for example, bind to a TDP and reduce substrate affinity, reduce substrate specificity, compete with a substrate for binding to TDP, or increase susceptibility of TDP to proteolysis.

As used herein, the term “decreasing” when used in reference to cellular proliferation is intended to mean effecting a reduction in the amount or rate of cell growth. Effecting a reduction in the amount or rate of unregulated cell growth in TDP containing cells is a specific example of decreasing cellular proliferation.

As used herein, the term “TDP-containing cell” is intended to refer to a cell having a measurable amount of TDP. A TDP-containing cell can contain an amount of TDP that is sufficient to perform normal repair function of TDP.

As used herein, the term “reducing the severity,” when used in reference to a proliferative disease, is intended to mean an arrest or decrease in clinical symptoms, physiological indicators or biochemical markers of proliferative disease. Clinical symptoms include perceptible, outward or visible signs of disease. Physiological indicators include detection of the presence or absence of physical and chemical factors associated with a process or function of the body. Biochemical markers include those signs of disease that are observable at the molecular level, such as the presence of a disease marker, such as a tumor marker. A tumor marker is a substance in the body that usually indicates the presence of cancer. Tumor markers are usually specific to certain types of cancer and are usually found in the blood or other tissue samples. One skilled in the art will be able to recognize specific clinical symptoms, physiological indicators and biochemical markers associated with a particular proliferative disease.

As used herein, the term “effective amount” when used in reference to reducing the severity of a proliferative disease, such as cancer, is intended to mean an amount of TDP inhibiting compound administered to an individual required to effect a decrease in the amount or rate of spread of a neoplastic condition or pathology. The amount of a TDP inhibiting compound required to be effective will depend, for example, on the type or types of TDP inhibiting compounds administered, the pathological condition to be treated and the level of abundance of TDP in a cell, as well as the weight and physiological condition of the individual, and previous or concurrent therapies. An amount considered as an effective amount for a particular application of TDP inhibiting compound will be known or can be determined by those skilled in the art, using the teachings and guidance provided herein. One skilled in the art will recognize that the condition of the patient can be monitored throughout the course of therapy and that the amount of the modulating compound that is administered can be adjusted according to the individual's response to therapy.

As used herein, the term “cancer” is intended to mean a class of diseases characterized by the uncontrolled growth of aberrant cells, including a variety of known cancers and neoplastic conditions, whether characterized as malignant, benign, soft tissue or solid tumor. Exemplary specific cancers include breast, ovarian, small-cell, leukemia, lymphoma, melanoma and prostate cancers.

The invention provides a TDP inhibiting compound. The structure of the compound is:

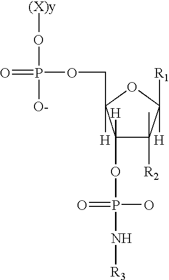

wherein,

X is a nucleotide;

Y is a positive integer;

R1 is any nucleotide base;

R2 is H; and

R3 is phenyl or substituted phenyl.

The polynucleotide-3′-bridging phosphoramidate of the invention can contain a variety of substituents that preferably function as poor leaving groups at the R3 position.

Exemplary polynucleotide-3′-phosphodiester derivatives and kᵣₑₗ values representing the rates of cleavage by human TDP, relative to 3′-paranitrophenyl, indicated in parentheses, include:

| | |
|---|---|
| aniline derivative (kᵣₑₗ = 0.05) | 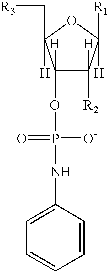 |
| toluidine derivative (kᵣₑₗ = 0.025) | 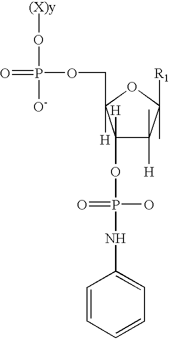 |
| N,N-dimethyl-1,4- phenylene-diamine derivative (kᵣₑₗ = 0.0125) | 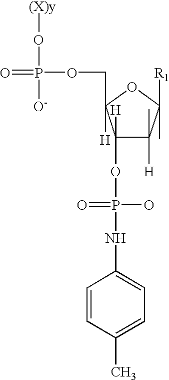 |
| Azidoaniline derivative (undetectable activity) | 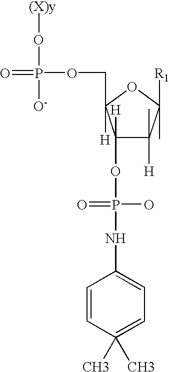 |

Other exemplary moieties which can be present in a polynucleotide-3′-bridging phosphoramidate, polynucleotide-3′alkylaphosphonate, polynucleotide 3′-phosphorothioate, polynucleotide-3′-alkyl phosphotriester, polynucleotide-bridging-alkyl phosphonate, nucleotide-3′-bridging phosphoramidate, nucleotide-3′-alkyl phosphonate, nucleotide-3′-alkyl phosphotriester, or nucleotide-bridging-alkyl phosphonate include:

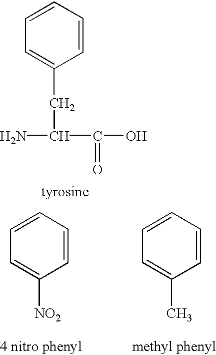

A polynucleotide-3′-bridging phosphoramidate of the invention can be conveniently prepared as described in Example III, by modifications of methods that produce the polynucleotide-3′ phosphodiester derivative compound of the invention, and by other methods which can be determined by those skilled in the art. Such modifications of the procedure for preparing a polynucleotide-3′phosphodiester derivative can include, for example, the use of alternate solvents, buffers and temperature conditions.

Methods for preparing a variety of polynucleotide-3′ derivatives are well known to those skilled in the art. Therefore, those skilled in the art will be able to determine methods for producing various polynucleotide-3′ phosphodiester derivatives, polynucleotide-3′ phosphoramidates, polynucleotide-3′-bridging phosphorothioates, polynucleotide-3′-alkyl phosphonates, polynucleotide-3′-alkyl phosphotriesters, polynucleotide-bridging-alkyl phosphonates, nucleotide-3′-bridging phosphoramidates, nucleotide-3′-alkyl phosphonates, nucleotide-3′-alkyl phosphotriesters, and nucleotide-bridging-alkyl phosphonates.

The invention provides a composition containing a TDP inhibiting compound, such as a polynucleotide-3′-bridging phosphoramidate aniline derivative, and a camptothecin. The term “camptothecin” as used herein is intended to mean a camptothecin or camptothecin derivative that functions as a topoisomerase I inhibitor. Exemplary camptothecins include, for example, topotecan, irinotecan, DX-8951f, SN38, BN 80915, lurtotecan, 9-nitrocamptothecin and aminocamptothesin. A variety of camptothecins have been described, including camptothecins used to treat human cancer patients. Several camptothecins are described, for example, in Kehrer et al., Anticancer Drugs, 12(2):89-105, (2001).

The invention provides modified nucleotides that inhibit TDP activity. Such modified nucleotides include, for example, nucleotide-3′-bridging phosphoramidates, nucleotide-3′-phosphorothioates, nucleotide-3′-alkyl phosphonates, and nucleotide-3′-alkyl phosphotriesters. Modified nucleotides can be prepared by those skilled in the art using the methods provided herein for preparing various modified polynucleotides. Alternative methods for preparing modified nucleotides are well known to those skilled in the art.

Exemplary modified nucleotides that inhibit TDP activity are:

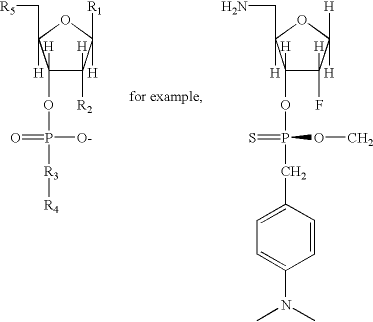

wherein R1 is a hydrogen atom or nucleotide base, R2 is a hydrogen atom, hydroxyl, azido, halo, amino, or O-alkyl; R3 is amino or methyl; R4 is phenyl or substituted phenyl; and R5 is a hydrogen atom, hydroxyl, azido, halo, amino, or O-alkyl.

The invention provides a method of inhibiting 3′-tyrosyl-DNA-phosphodiesterase (TDP) activity, comprising contacting a TDP with a TDP inhibiting compound, such as a polynucleotide-3′-bridging phosphoramidate aniline derivative.

A TDP can be contained in a variety of samples, as well as in a cell, tissue or organ of an individual. A TDP can be present in, for example, a fluid or tissue obtained from an animal, a cell obtained from an animal fluid or tissue, cultured cells, recombinant cells or organisms expressing TDP, and lysates or fractions thereof. A TDP can also be contained in a purified preparation.

The invention provides a method of decreasing cellular proliferation, comprising contacting a TDP-containing cell with an effective amount of a TDP inhibiting compound sufficient to inhibit TDP activity in said cell.

A TDP inhibiting compound useful in the method of decreasing cellular proliferation can be, for example, a polynucleotide-3′ phosphodiester derivative, polynucleotide-3′-bridging phosphoramidate, a polynucleotide-3′-phosphorothioate, a polynucleotide-3′-alkyl phosphonate, polynucleotide-3′-alkyl phosphotriester, polynucleotide-bridging-alkyl phosphonate, nucleotide-3′-bridging phosphoramidate, nucleotide-3′-alkyl phosphonate, nucleotide-3′-alkyl phosphotriester, nucleotide-bridging-alkyl phosphonate or a combination thereof. Exemplary polynucleotide-3′-bridging phosphoramidates useful in the methods of the invention for decreasing cellular proliferation are polynucleotide-3′-bridging phosphoramidate aniline and toluidine derivatives.

The methods of the invention for decreasing cell proliferation can be applied to a variety of TDP-containing cells. For example, it can be desirable to decrease cell proliferation in normal or pathologically aberrant cells. Pathologically aberrant cells can be for example, cells having uncontrolled cell growth. Such hyperproliferative cells include, for example, neoplastic cells and cancer cells including ovarian, small-cell, breast, leukemia, lymphoma and other types of cancer cells. A variety of methods can be used to determine if TDP is expressed in a particular cell, such as a cancer cell. Such methods are well known to those skilled in the art and include immunological methods such as ELISA, Western blotting, RIA, immunofluorescence detection methods, methods based on protein or peptide chromatographic separation, methods based on characterization of TDP mRNA and mass spectrometric detection.

The ability of a TDP modulating compound to alter TDP activity can be determined, for example, using 3′-(4-nitro-phenyl)-oligonucleotides, which provide a convenient calorimetric readout of phosphodiesterase activity, as described in Example II. The effect of a TDP modulating compound on TDP activity can also be tested using 3′-tyrosyl oligonucleotides and phosphorothioate trapped topoisomerase-DNA complexes. A variety of topoisomerase-DNA complexes can be prepared using methods described in Burgin et al., Nuc. Acids Res. 23:29873-2979, (1995), for example. If desired, for cell-based assay of TDP activity, the concentration of topoisomerase-DNA complex can be modulated in a cell, for example, using topoisomerase poisons such as camptothecins, topoisomerase mutants having altered DNA binding characteristics and by altering topoisomerase expression level in a cell.

Inhibition constants (K_{I}) can be defined by measuring the rate of TDP cleavage as a function of inhibitor concentration. The specificity of the inhibitors can be determined, for example, by assaying the ability of the inhibitors to slow or prevent tyrosine phosphatase activity or topoisomerase I DNA relaxation activity.

The effect of a TDP inhibiting compound on a neoplastic or cancer cell can be assessed by several criteria well known in the art. For example, a neoplastic or cancer cell can be distinguished from a normal cell by the uncontrolled growth and invasive properties characteristic of cancer cells. Using histological methods, a cancer cell can be observed to invade into surrounding normal tissue, have an increased mitotic index, and increased nuclear to cytoplasmic ratio, altered deposition of extracellular matrix, and a less differentiated phenotype. The unregulated proliferation of a cancer cell can be characterized by anchorage independent cell growth, proliferation in reduced-serum medium, loss of contact inhibition, and rapid proliferation compared to normal cells. Those skilled in the art will know how to determine if a TDP inhibiting compound is effective in promoting a more normal phenotype in a cancer cell. Those skilled in the art will also be able to detect a cancer cell in a population of cells, tumor, or organ.

Animal models of hyperproliferative diseases similarly can be used to assess the activity of TDP inhibiting compound or an amount sufficient to inhibit the activity of TDP in a cell. Animal models of such pathological conditions well known in the art which are reliable predictors of treatments in humans include, for example, animal models for tumor growth and metastasis and autoimmune disease.

Animal tumor models are known in the art which are predictive of the effects of therapeutic treatment. These models generally include the inoculation or implantation of a laboratory animal with heterologous tumor cells with simultaneous or subsequent administration of a therapeutic treatment. The efficacy of the treatment is determined by measuring the extent of tumor growth or metastasis. Measurement of clinical or physiological indicators can alternatively or additional be assessed as an indicator of treatment efficacy. Exemplary animal tumor models can be found described in, for example, Brugge et al. Origins of Human Cancer, Cold Spring Harbor Laboratory Press, Plain View, N.Y., (1991).

A variety of different classes of enzyme inhibitors can be used to inhibit TDP. For example, competitive inhibitors are designed to inhibit TDP by occupying the active site and preventing cleavage. Irreversible inhibitors are designed to place a chemically reactive group within the active site that can inactivate the enzyme. Mechanism-based inhibitors also generate reactive species within the active site, however the reactive groups are generated as a result of the cleavage reaction and should therefore be much more specific. Finally, small molecule inhibitors can be identified using a positional scanning method described herein.

One class of TDP inhibitors are nonhydrolyzable analogs. A nonhydrolyzable analog that binds tightly enough to the enzyme can function as a competitive inhibitor if the rate of disassociation (k_{off}) is sufficiently slow. Such analogs can be converted into nonhydrolyzable substrates by modifying the phosphodiester linkage between the DNA and the tyrosine residue (Wang et al., Med. Res. Rev. 17:367-425 (1997)). Bridging phosphoramidates have been demonstrated to be efficient nuclease inhibitors (Bannwarth, W. Helv. Chem. Acta 71:1517-1527 (1988)), and it has been shown that 5′-bridging phosphoramidate linkages prevent Topo I-mediated cleavage (Burgin, A., Huizenga, B., and Nash, H. Nuc. Acids Res. 23, 2973-2979 (1995)).

A second method for preventing phosphodiester hydrolysis and transesterification reactions is to substitute one of the non-bridging oxygens of the phosphodiester (Eckstein, F. Oligonucleotides and Their Analogs. (IRL Press) (1992)). Reagents useful for synthesizing phosphorothioate (Yang, S. et al., Proc. Natl. Acad. Sci. 93:11534-11539 (1996)) and methyl phosphonate analogs (Burgin, A. et al., Nuc. Acids Res. 23:2973-2979 (1995)) are commercially available (Glenn Research, Sterling, Virginia). These reagents can be conveniently used with tyrosine derivatized resins to yield the desired products. The phosphorothioate and phosphonate analogs can also be synthesized with a bridging phosphoramidate linkage to form a competitive inhibitor. A variety of different functionalities can be placed at the para position of the aromatic ring. For example, a chemically reactive group can be placed within the active site of the enzyme by positioning at the para position of the aromatic ring. If such a group reacts faster than the rate of dissociation, the inhibitor will become irreversibly linked to the enzyme. For example, an aziridine group placed at the para position places very reactive electrophile within the active site of the enzyme. The greatest difficulty with this approach is placing the aziridine functionality within a deprotected oligonucleotide. A strategy based on the method of convertible bases has been previously developed to place aziridine functions at the 4 position of thymidine (Zheng et al., Nucleos. Nucleot. 14:939-942 (1995)) within an oligonucleotide, and this technology can be applied to modify the 4 position (para) of 3′-phospho-phenyl oligonucleotides (see Example IV).

The third class of substrate analogs are designed to act as mechanism-based inhibitors. These analogs are similar to the irreversible inhibitors described above, however in this class of molecules a chemically reactive group is generated as a result of the cleavage reaction itself (Silverman, R. Mechanism-Based Enzyme inactivation: Chemistry and Enzymology (CRC Press) (1988)). This feature can dramatically increase the specificity of the inhibitor (see Example V).

The invention provides a method of screening for compounds that modulate the activity of TDP. The method comprises (a) contacting TDP, or a fragment or modification thereof having TDP activity, with a compound under conditions that allow TDP activity, (b) determining an amount of TDP activity, and (c) identifying a compound that modulates the activity of TDP, or a fragment of modification thereof.

A TDP to be contacted in the methods of the invention for screening for compounds that modulate the activity of TDP can be contained in a variety of sample types. For example, a TDP can be contained in an animal, including a human, such as when in vivo or in situ detection methods are employed, as well as in samples obtained or derived from the animal, such as when ex vivo detection methods are employed. A TDP can also be contained in a cell that recombinantly expresses TDP and in a lysate or fraction of such a cell. A TDP can be contained in a histologic section of a specimen obtained by biopsy, cells obtained from body fluids, or cells that are placed in or adapted to tissue culture. An isolated TDP is removed or separated from at least one component with which it is naturally associated. Therefore, an isolated TDP can be contained in a subcellular fraction or extract prepared from such cells, such as a cytoplasmic lysate, a membrane preparation, a nuclear extract, or a crude or purified protein preparation. A sample containing a TDP can be prepared by methods known in the art suitable for the particular format of the detection method. For example, biochemical methods such as precipitation, chromatography and immunoaffinity methods can be used to isolate a TDP from a cell which expresses TDP endogenously or recombinantly. Procedures for preparing subcellular fractions, such as nuclear fractions, and cell lysates are well known to those skilled in the art, and include, for example, cell disruption followed by separation methods such as gradient centrifugation and biochemical purification methods. An exemplary method for purifying TDP is described herein, in Example I.

An amount of TDP activity can be determined using a variety of methods. For example, the amount or rate of TDP activity can be determined using polynucleotide and non-polynucleotide substrates (see Example II). Exemplary assays for detecting 3′-tyrosyl-phosphodiesterase activity qualitatively and quantitatively are provided herein, in Example II. Other methods for determining phosphodiesterase activity well known to those skilled in the art can be used for determining an amount of TDP activity. The amount of TDP mRNA or polypeptide contained in a cell, tissue or organ indicates the amount of TDP activity present in the particular sample, and can therefore be used as a qualitative or quantitative indication of the amount of TDP activity in the sample. Methods for determining the amount of TDP mRNA and polypeptide amounts in a cell, tissue or organ, using in situ and in vitro methods well known to those skilled in the art can be used to determine an amount of TDP activity in a cell, tissue or organ.

As understood by those of skill in the art, assay methods for identifying compounds that modulate TDP activity generally require comparison to a control. An exemplary control is a cell or isolated TDP preparation that is treated substantially the same as the test cell or preparation exposed to a compound, except that the control is not exposed to the compound. A control cell or isolated TDP preparation can be treated with a carrier solution or solvent in which a compound is dissolved or contained, such as an aqueous or organic solution, if desired.

A compound identified using the methods of the invention can modulate TDP activity by a variety of mechanisms. For example, a compound can act directly by binding to TDP and altering a function, such as enzyme activity or substrate affinity or avidity, or can act indirectly by binding to a molecule that alters TDP activity. A molecule that alters TDP activity can function, for example, by modulating the amount of TDP expressed or contained in a cell. A compound can act to decrease TDP activity by decreasing the amount of TDP polypeptide in a cell, for example, by stimulating decreased TDP mRNA expression. TDP mRNA expression can be decreased, for example, by inducing or derepressing the transcription of a TDP gene and by regulating the expression of a cellular protein that acts as a transcription factor to regulate gene expression. A compound can act to decrease the amount of TDP activity by decreasing the stability of a TDP mRNA or polypeptide, for example, by increasing a cellular degradation activity, such as a protease activity. Conversely, a compound can act to increase an amount of TDP activity.

A TDP can be recombinantly expressed in a variety of cell types using well known expression systems and methods, such as those described in Sambrook et al., Molecular Cloning: A Laboratory Manual, 3rd ed., Cold Spring Harbor Press, Plainview, N.Y. (2001)).

Compounds useful as potential therapeutic agents can be generated by methods well known to those skilled in the art, for example, well known methods for producing pluralities of compounds, including chemical or biological molecules such as simple or complex organic molecules, metal-containing compounds, carbohydrates, peptides, proteins, peptidomimetics, glycoproteins, lipoproteins, nucleic acids, antibodies, and the like, are well known in the art and are described, for example, in Huse, U.S. Pat. No. 5,264,563; Francis et al., Curr. Opin. Chem. Biol. 2:422-428 (1998); Tietze et al., Curr. Biol., 2:363-371 (1998); Sofia, Mol. Divers. 3:75-94 (1998); Eichler et al., Med. Res. Rev. 15:481-496 (1995); and the like. Libraries containing large numbers of natural and synthetic compounds also can be obtained from commercial sources. Combinatorial libraries of molecules can be prepared using well known combinatorial chemistry methods (Gordon et al., J. Med. Chem. 37: 1233-1251 (1994); Gordon et al., J. Med. Chem. 37: 1385-1401 (1994); Gordon et al., Acc. Chem. Res. 29:144-154 (1996); Wilson and Czarnik, eds., Combinatorial Chemistry: Synthesis and Application, John Wiley & Sons, New York (1997)). Example VI provides an exemplary small molecule library which can be screened to identify a TDP modulating compound, and Example VII provides an exemplary method for deconvoluting such a small molecule library.

Such libraries can be screened to identify a compound that modulates TDP amount or activity using assay methods described herein, such as the spectrophotometric and fluorescence-based assays described in Example II. The effectiveness of compounds identified by an initial in vitro screen can be further tested in vivo using animal models of proliferative disease well known in the art. However, if desired, compounds can be screened using an in vivo assay.

Compounds identified as methods of the invention can be administered to an individual, for example, to alleviate a sign or symptom of a proliferative disease, such as cancer. Such compounds are useful therapeutic agents. One skilled in the art will know or can readily determine the alleviation of a sign or symptom associated with cancer, such as ovarian cancer, as methods for diagnosing and staging cancer are well known to those skilled in the art.

The invention provides a method of reducing the severity of a proliferative disease. The method involves administering to an individual an effective amount of a TDP inhibiting compound sufficient to inhibit the activity of TDP in cancer cells in the individual, thereby decreasing cell proliferation to reduce the severity of a proliferative disease.

The TDP modulating compounds of the invention can be formulated and administered by those skilled in the art in a manner and in an amount appropriate for the condition to be treated; the weight, gender, age and health of the individual; the biochemical nature, bioactivity, bioavailability and side effects of the particular compound; and in a manner compatible with concurrent treatment regimens. An appropriate amount and formulation for decreasing cell proliferation in humans can be extrapolated based on the activity of the compound in the assays described herein. An appropriate amount and formulation for use in humans for other indications can be extrapolated from credible animal models known in the art.

The total amount of compound can be administered as a single dose or by infusion over a relatively short period of time, or can be administered in multiple doses administered over a more prolonged period of time. Additionally, the compounds can be administered in slow-release matrices, which can be implanted for systemic delivery or at the site of the target tissue. Contemplated matrices useful for controlled release of therapeutic compounds are well known in the art, and include materials such as DepoFoam™, biopolymers, micropumps, and the like.

The compounds and compositions of the invention can be administered to the subject by any number of routes known in the art including, for example, intravenously, intramuscularly, subcutaneously, intraorbitally, intracapsularly, intraperitoneally, intracisternally, intra-articularly, intracerebrally, orally, intravaginally, rectally, topically, intranasally, or transdermally. A preferred route for humans is oral administration.

A TDP modulating compound can be administered to a subject as a pharmaceutical composition comprising the compound and a pharmaceutically acceptable carrier. Those skilled in the art understand that the choice of a pharmaceutically acceptable carrier depends on the route of administration of the compound and on its particular physical and chemical characteristics. Pharmaceutically acceptable carriers are well known in the art and include sterile aqueous solvents such as physiologically buffered saline, and other solvents or vehicles such as glycols, glycerol, oils such as olive oil and injectable organic esters.

A pharmaceutically acceptable carrier can contain physiologically acceptable compounds that stabilize the compound, increase its solubility, or increase its absorption. Such physiologically acceptable compounds include carbohydrates such as glucose, sucrose or dextrans; antioxidants, such as ascorbic acid or glutathione; chelating agents; and low molecular weight proteins.

For applications that require the compounds and compositions to cross the blood-brain barrier, formulations that increase the lipophilicity of the compound are particularly desirable. For example, a TDP modulating compound can be incorporated into liposomes (Gregoriadis, Liposome Technology, Vols. I to III, 2nd ed. (CRC Press, Boca Raton Fla. (1993)). Liposomes, which consist of phospholipids or other lipids, are nontoxic, physiologically acceptable and metabolizable carriers that are relatively simple to make and administer.

In current cancer treatment, more than one compound is often administered to an individual for maximal reduction in symptoms. Thus, for use in treating cancer, a TDP modulating compound can be formulated with a second compound used for treating cancer. Compounds used for treating proliferative disease include, for example, cisplatin, flurouracil, methotrexate, ifosfamide, mitoxantrone, paclitaxel and camptothecins, such as Topotecan. Many of these drugs are administered to cancer patients at relatively high levels and adverse effects are common. Adverse side effects can be minimized by increasing the potency of a compound. Therefore, a TDP inhibiting compound can be combined with such compounds to effectively reduce symptoms in individuals having proliferative diseases such as cancer. A compound of the invention can be combined, for example, with a camptothecin. Types of camptothecins, formulations, routes of delivery and dosing of camptothecins are well known and are described, for example, in Kehrer et al., Anticancer Drugs, 12(2):89-105, (2001).

Camptothecin (CPT) family cytotoxic agents specifically inhibit topoisomerase I (Topo I). A number of experiments indicate that the actual target of camptothecins is the covalent 3′-phosphotyrosyl Topo I-DNA intermediate and that these poisons specifically inhibit the ligation step. First, the poisons do not appear to affect the rate of cleavage in vitro. However, CPT increases the yield of covalent intermediates when reactions are stopped with SDS. These results are consistent with a shift in the pseudo-equilibrium between cleavage and ligation because of a decrease in the rate of ligation. In addition, camptothecin and its derivatives bind to the covalent Topo I-DNA intermediate although the binding interactions are not well defined. Finally, CPT rapidly blocks both DNA and RNA synthesis in treated cells and is highly S-phase specific. Taken together, the data argues that stabilization of the covalent enzyme-DNA intermediate converts Topo I into a DNA damaging agent. Additional data demonstrates that this damage leads to the formation of persistent double strand breaks, that in turn trigger apoptosis or G-phase cell cycle arrest. Several other known Topo I poisons, including coralyne and Hoeschst 33342 also impede the religation step of catalysis and mediate cell killing by similar mechanisms. Cell killing by these compounds is caused by an accumulation of topoisomerase-DNA complexes which overwhelm the ability of the cell to repair the covalent complexes. Inhibition of the repair enzyme TDP will further increase the efficacy of such topoisomerase poisoning compounds in cell killing.

It is understood that modifications which do not substantially affect the activity of the various embodiments of this invention are also included within the definition of the invention provided herein. Accordingly, the following examples are intended to illustrate but not limit the present invention.

### EXAMPLE I

### Purification of 3′-Tyrosyl-DNA-Phosphodiesterase

This example shows purification of calf thymus 3′-tyrosyl-DNA-phosphodiesterase.

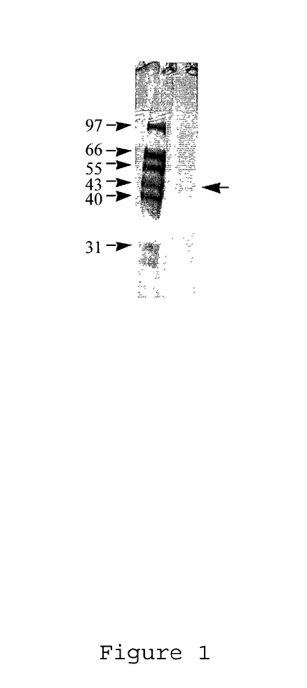

3′-tyrosyl-DNA-phosphodiesterase (TDP) was purified from calf thymus. A summary of the purification is provided in Table 1, below. The activity was difficult to detect after initial sonication and solubilization of the calf thymus but was detectable after the first purification step. The results demonstrate that the final preparation has a >300,000-fold greater specific activity over the starting crude lysate. Polyacrylamide gel analysis of the final enzyme preparation also indicates that a single dominant polypeptide is visible following Coomassie blue staining of the gel (two lanes from the same SDS polyacrylamide gel are shown in FIG. 1). Two fainter bands are present at approximately 60 and 65 KD. The data presented in Table 1 and FIG. 1 demonstrate that the current preparation is pure.

TABLE 1

| | mg | | |
|---|---|---|---|
| | protein | Units PDE* | Units/mg |
| Crude Lysate | 910 | 5 | 0.005 |
| Ion Exchange | 3.1 | 360 | 120 |
| Ion Exchange | 0.5 | 550 | 1,100 |
| Size Exclusion | 0.3 | 400 | 1,600 |

*One unit of enzyme cleaves 1 umol of substrate in one minute

Thus, TDP can be purified from a mammalian tissue using biochemical methods.

### EXAMPLE II

### 3′-Tyrosyl-DNA-Phosphodiesterase Substrates and Activity Assays

This example shows TDP activity assays. DNA containing 5′-bridging phosphorothioate linkages are efficiently cleaved by Topoisomerase I. However, the cleavage generates a 5′-sulfhydryl (instead of a 5′-OH) that is an ineffective in subsequent ligation reactions thereby trapping the enzyme-DNA covalent intermediate (Burgin, et al. Nuc. Acids Res. 23:2973-2979 (1995)). TDP catalyzes the specific removal of topoisomerase I from this suicide DNA. Substrates containing only a single tyrosine linked to the 3′-end of DNA, were prepared. One type of substrate made use of a tyrosine residue present on a resin suitable for oligonucleotide synthesis. Different resins can be prepared in order to place different tyrosine analogs onto the 3′-end of the DNA. Following standard phosphoramidite synthesis and deprotection, a single tyrosine residue remained present on the 3′-end of the DNA. These substrates are cleaved efficiently by TDP.

Synthesis of competitive and irreversible inhibitors can be performed by placing different tyrosine analogs onto the 3′-end of DNA. Therefore, a post-synthetic approach for placing different analogs onto the 3′-end of DNA was developed. One substrate useful in a variety of convenient assay formats is 4-nitro-phenyl derivatized DNA. This substrate is particularly useful because cleavage by TDP generates 4-nitro-phenol, which absorbs at a unique wavelength (400 nm). This feature enables the assay to be detected spectrophotometrically. This is useful because the assays can be carried out in 96-well plates and the rate of cleavage can be measured in real time.

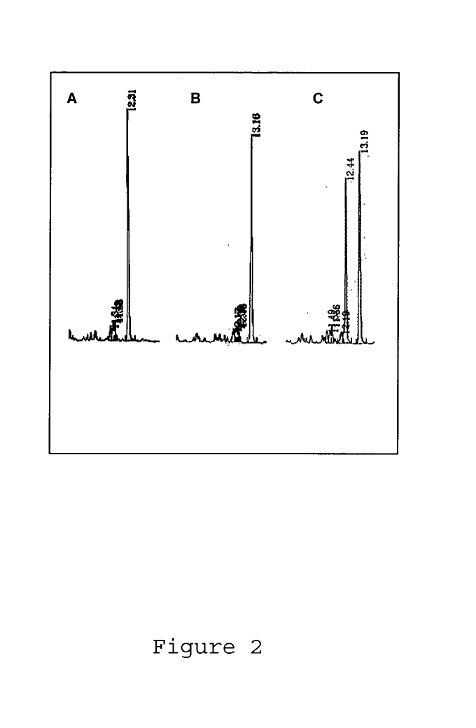

Oligonucleotides containing a 3′-monophosphate were synthesized. These oligonucleotides were purified and the 5′-dimethoxytrityl group was not removed in order to protect the 5′-OH. A water soluble condensing reagent (1-(-3-dimethylaminopropyl)-3-ethylcarbodiimide; EDC) was then used to link 4-nitro-phenol to the 3′-phosphate group (Shabarova, Z. Biochimie 70:1323-1334 (1988)). The yield from this reaction was generally greater than 80%. In addition, the products were easily purified by reverse phase HPLC, as shown in FIG. 2. FIG. 2 shows that polynucleotide 17-mers labeled with EDC and 4-nitro-phenol can be detected by reverse phase HPLC. HPLC was performed using a Biorad Spherisorb column with 100 mM TEAA, 5-25% acetonitrile at 1 ml/min at 55° C. The underivatized polynucleotide had a retention time of 12.31 minutes (panel A). Incubation with EDC and 4-nitro-phenol caused the polynucleotide to elute at 13.15 minutes (panel B). A coinjection of underivatized and derivatized material demonstrated that the change in retention time is significant and reliable (panel C). This approach was used to derivatize polynucleotides with tyrosine, phenol, serine, aniline, and 4-nitro-aniline. These results demonstrate that a large number of different 3′-derivatized polynucleotides can be easily synthesized and purified.

The purified TDP does not cleave tyrosine analogs linked through a 5′-phosphodiester linkage. In addition, the enzyme does not degrade 5′-3′-DNA or -RNA phosphodiester linkages. Therefore, TDP activity was specific for 3′-tyrosyl-phosphodiesters. Similar results were observed for TDP purified from yeast (Yang, et al Proc. Natl. Acad. Sci. 93:11534-11539,(1996)).

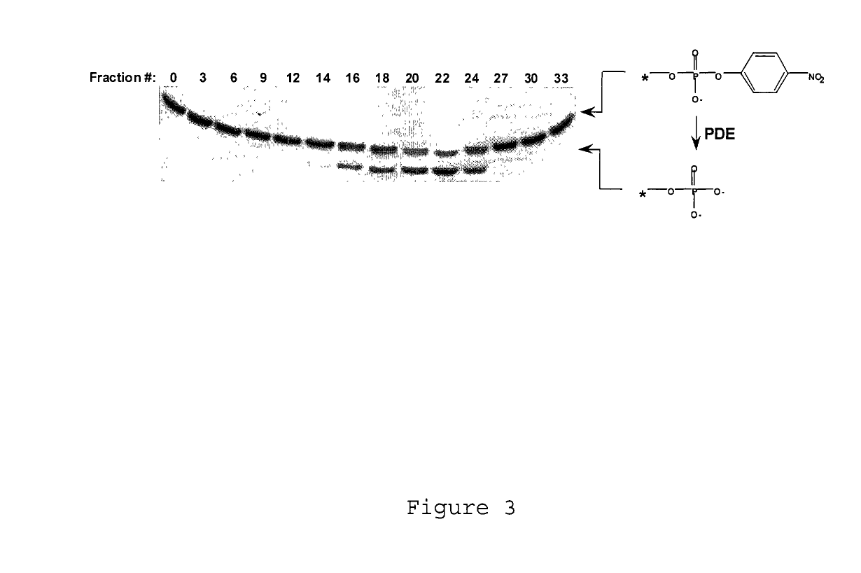

TDP cleavage assays were detected by 5′-end labeling the 3′-derivatized oligonucleotides. In a typical reaction, TDP enzyme and oligonucleotides were incubated at 37° C. and then quenched with SDS (0.1%) and urea (5M final concentration). Reaction products were then resolved on a denaturing polyacrylamide sequencing gel (0.5×TBE, 0.5 mm gel, 15% acrylamide, 8M urea). The phosphodiesterase reaction generated an oligonucleotide containing a 3′phosphate, but lacking the 3′-tyrosine analog (4-nitro-phenol), which migrates faster within the gel than unreacted oligonucleotide. FIG. 3 shows the results oligonucleotide cleavage by TDP activity within individual fractions from a column purification. TDP activity was present in fractions 14-24.

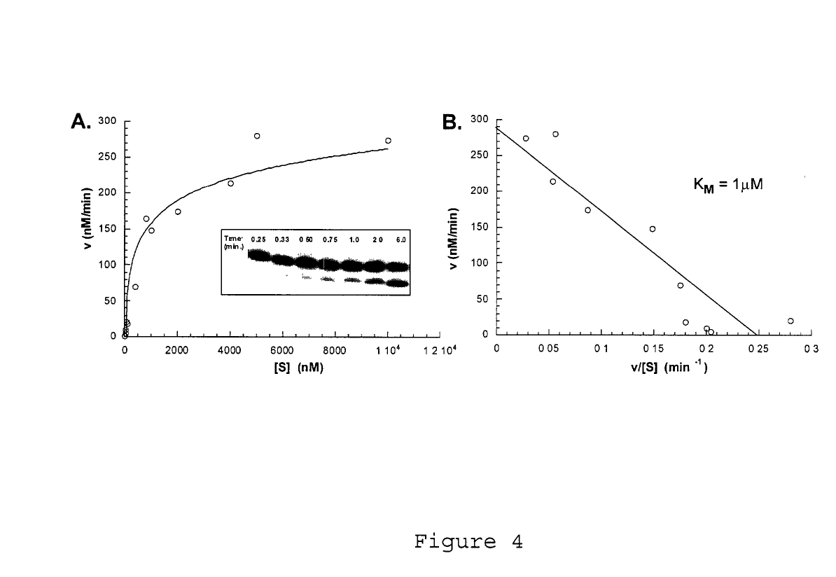

The amount of cleavage can be accurately quantitated by phosphorimager analysis of a dried gel, allowing kinetic analysis of the enzyme. Reaction velocities were measured as a function of substrate concentration. In FIG. 4A, the reaction velocity (nM/min) was plotted as a function of substrate concentration (nM), resulting in a hyperbolic curve. The same data is plotted in FIG. 4B (Eadie-Hofstee plot), which indicates an apparent KM value of 1 mM.

The enzyme also efficiently cleaved a tyrosine analog when the substrate is duplex DNA. Data suggest that the tyrosine analog was cleaved efficiently (increased kcat/KM) when the substrate is present within a DNA duplex.

These results also demonstrate the ability to accurately measure small differences in reaction velocities/rates. This feature is useful for testing inhibitors.

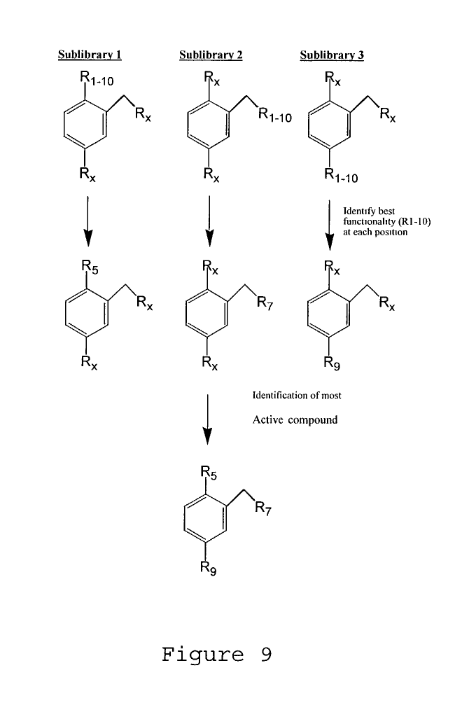

TDP activity can be conveniently monitored by a variety of spectrophotometric and fluorescence-based assays. FIG. 9 shows the chemical structure of a TDP substrate useful for spectrophotometric detection of TDP activity. Cleavage of the substrate, shown on the left in FIG. 9, by TDP results in the formation of para-nitrophenol. Para-nitrophenyl absorbs UV light at 405 nm and as such, the amount of para-nitrophenyl present in a sample can be conveniently detected visually or using a spectrophotometer. A variety of substrates can be cleaved by TDP to form a detectable product. For example, a variety of substitutions can be made in the substrate shown in FIG. 9. In particular, the bridging oxygen in para-nitrophenyl can be replaced with sulfur or nitrogen, to generate 4-nitro-thiophenyl or 4-nitroaniline, shown below, which can be detected spectrophotometrically.

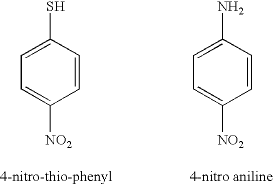

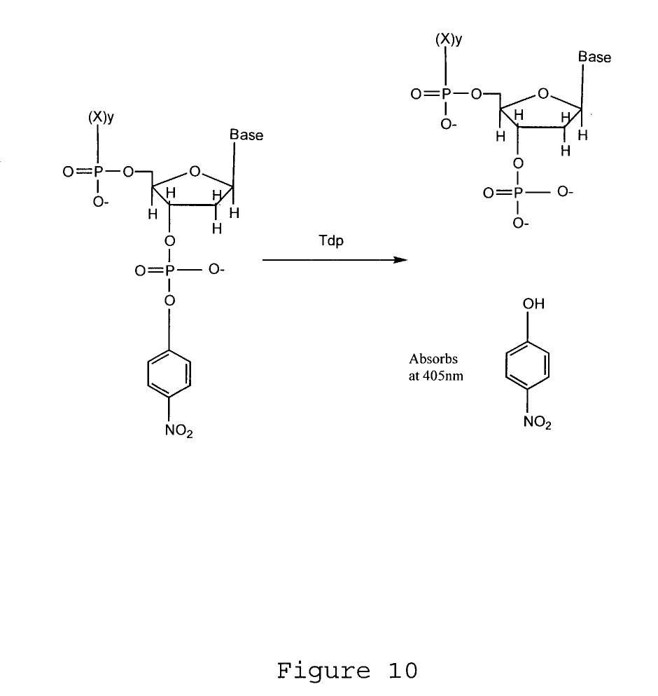

FIG. 10 shows the chemical structures of a TDP substrate useful in a fluorescence-based TDP activity assay, and the corresponding fluorescent product. In a TDP assay employing the substrate shown in FIG. 10, the para-nitrophenyl moiety acts as a quencher of a fluorescent base, etheno adenosine, which fluoresces upon excitation at about 300 nm. Prior to cleavage by TDP, the para-nitophenyl moiety absorbs 300 nm light and prevents maximal excitation of the etheno adenosine moiety. Upon TDP cleavage of the substrate, the para-nitrophenyl moiety is released from the substrate molecule, and fluorescence of the etheno adenosine moiety increases. Thus, TDP activity can be detected by measuring the formation of the fluorescent product. A variety of fluorescent bases or other detectable moieties can be incorporated into such a substrate at the position indicated in FIG. 10, as well as at additional or other positions within a substrate molecule. Additionally, a variety of moieties in addition to para-nitrophenyl can serve to quench the fluorescence of a selected fluorescent base or moiety.

Thus, TDP activity can be determined using a variety of polynucleotide and non-polynucleotide substrates, both qualitatively and quantitatively. Quantitative methods can be used to determine kinetic parameters for characterizing the ability of a compound to modulate TDP activity.

### EXAMPLE III

### Synthesis of 3′-Tyrosine-DNA Phosphodiesterase Inhibitors

This example shows that TDP inhibitors can be synthesized via a condensation reaction using commercially availably reagents.

Polynucleotide derivative TDP inhibitors can be synthesized using the following methods. First, an oligonucleotide of 16 bases was synthesized on an ABI 392 DNA synthesizer using 3′-phosphate controlled pore glass resin (Glen Research, Sterling, Virginia) to build the DNA upon. The resultant oligo has a 3′ phosphate upon which the various functional groups are condensed. There are three different functional groups:

The Aniline derivative:

100-200 uM of a 16 base oligonucleotide with a 3′ phosphate in 200 uL of water

25 uL of MES at pH 5.5 as a buffer

1 uL of 0.1 M MgCl

22.8 uL of Aniline

0.024 g of 1-[3-(Dimethylamino)propyl]-3ethylcarbodiimide hydrochloride 98+%

The above reagents were mixed in an eppendorf tube and shaken vigorously for 2 hours. The DNA was then precipitated using ethanol and 3M sodium acetate, and then re-suspended in 600 uL of water. The 600 uL re-suspension was then purified by high-pressure liquid chromatography via an anion exchange column (DIONEX).

The Toulidine derivative:

100-200 uM of a 16 base oligonucleotide with a 3′ phosphate in 200 uL of water

25 uL of MES at pH 5.5 as a buffer

1 uL of 0.1 M MgCl

0.0268 g of p-Toluidine

0.024 g of 1-[3-(Dimethylamino)propyl]-3-ethylcarbodiimide hydrochloride 98+%

The above reagents were mixed in an eppendorf tube. This mixture were warmed and vortexed briefly in order to get the p-Toluidine to go into solution (90° C. for 1 min). After the p-Toluidine dissolved, the mixture was shaken vigorously for 2 hours. The DNA was then precipitated using ethanol and 3M sodium acetate, and then re-suspended in 600 uL of water. The 600 uL re-suspension was then purified by high-pressure liquid chromatography via an anion exchange column (DIONEX).

The N′N-Dimethyl-1,4-phenylene-diamine derivative:

100-200 uM of a 16 base oligonucleotide with a 3′ phosphate in 250 uL of water

250 uL of acetonitrile

25 uL of MES at pH 5.5 as a buffer

1 uL of 0.1 M MgCl

0.03405 of N′N-Dimethyl-1,4-phenylene-diamine 97%

0.024 g of 1-[3-(Dimethylamino)propyl]-3-ethylcarbodiimide hydrochloride 98+%

The above reagents were mixed in an eppendorf tube. This mixture had acteonitrile as one half the total volume since the N′N-Dimethyl-1,4-phenylene-diamine is not soluble in water. This mixture was shaken vigorously for 2 hours once everything was in solution. Two extractions were then done with acetonitrile. The DNA was then precipitated using ethanol and 3M sodium acetate, and then re-suspended in 600 uL of water. The 600 uL re-suspension was then purified by high-pressure liquid chromatography via an anion exchange column (DIONEX).

The Azidoaniline derivative:

100-200 uM of a 16 base oligonucleotide with a 3′ phosphate in 200 uL of water

25 uL of MES at pH 5.5 as a buffer

1 uL of 0.1 M MgCl

0.022 g of 4-Azidoaniline hydrochloride 97%

0.024 g of 1-[3-(Dimethylamino)propyl]-3-ethylcarbodiimide hydrochloride 98+%

The above reagents were mixed in an eppendorf tube, and the eppendorf tube was wrapped in aluminum foil to keep reduce the exposure to UV light. The tube was then shaken vigorously for 2 hours. The DNA was then precipitated using ethanol and 3M sodium acetate, and then re-suspended in 600 uL of water. The 600 uL re-suspension was then purified by high-pressure liquid chromatography via an anion exchange column (DIONEX).

Thus, 3-derivatized polynucleotides can be produced using commercially available reagents.

### EXAMPLE IV

### Synthesis of Polynucleotides Containing a 3′ Phenyl Derivative

This example shows an exemplary method for synthesizing polynucleotides having 3′ phenyl derivatives.

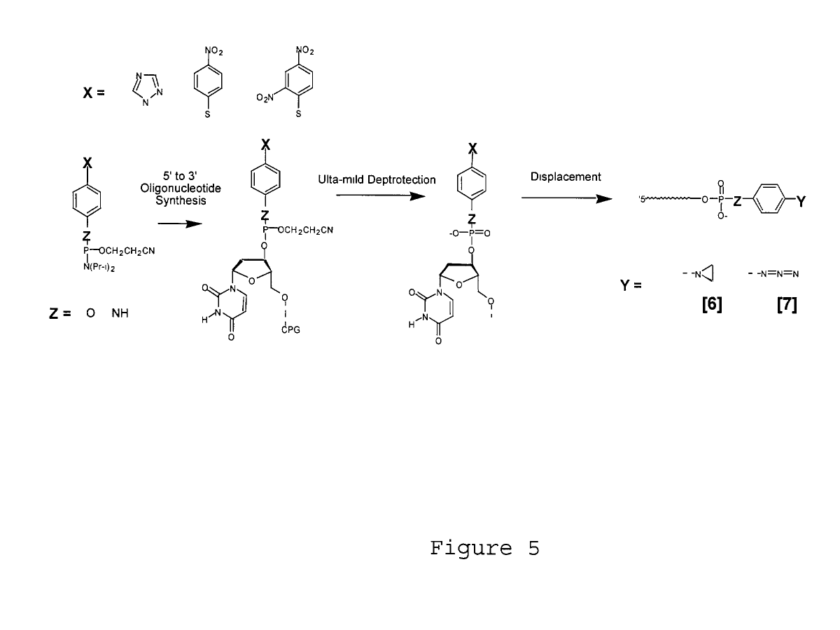

In FIG. 5, a starting phosphoramidite is diagrammed on the left. The polynucleotides were synthesized in the non-standard 5′ to 3′ direction. The para position (“X” in FIG. 5) can be substituted with a functionality that can be replaced following synthesis and deprotection of the oligonucleotide. Following deprotection, an aziridine ion displaces X, resulting in an oligonucleotide containing a 3′-phospho-(p-azido)-phenyl group (Yeh, et al. J. Biol. Chem. 269, 15498-15405, (1994)). This method has also been used to place photo-reactive groups within oligonucleotides (Zheng, et al. Nucleos. Nucleot. 14, 939-942, (1995)). For example, an azido ion can be used to produce polynucleotides containing 3′-phospho-(p-aziridine)-phenyl groups (Lanza, et al., J. Biol. Chem. 271, 6978-6986, (1996)). For both of these inhibitors, it can be advantageous to prevent chemical reactions so that the enzyme-substrate complex is long-lived enough to allow crosslinking. This can be accomplished by synthesizing the azido and aziridine analogs with the bridging phosphoramidate linkages (Z═NH) described above.

Thus, a polynucleotide containing a 3′ phenyl derivative can be prepared by the described methods.

### EXAMPLE V

### Mechanism-Based TDP Inhibitors

This example describes the rational for, and development of, mechanism-based TDP inhibitors.

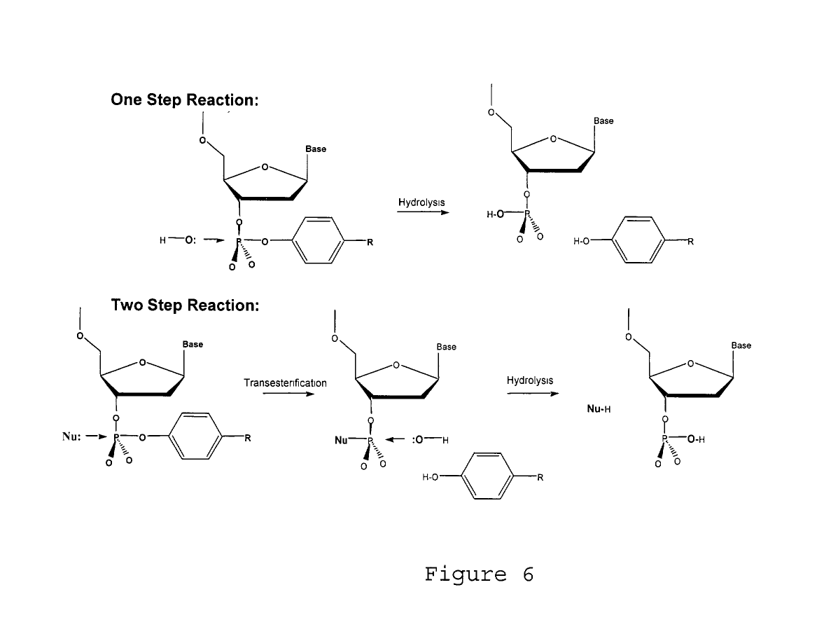

The phosphodiesterase reaction generates DNA containing a 3′-phosphate and free tyrosine (Yang, S., et al. Proc. Natl. Acad. Sci. 93, 11534-11539, (1996)). Two potential mechanisms would result in these reaction products. In the simplest reaction, water (diagrammed as a hydroxide ion; for simplicity, individual proton donors and acceptors are not detailed in FIG. 6) attacks the phosphodiester bond displacing tyrosine. This simple hydrolysis reaction would result in DNA containing a 3′-phosphate and free tyrosine products. However, these same products can result from a two step reaction. In the first step, an enzyme-bound nucleophile (Nu: in FIG. 6) can attack the phosphodiester generating a covalent 3′-enzyme-DNA complex. Subsequent hydrolysis of this intermediate would result in the same products. This mechanism is similar to the mechanism used by tyrosine phosphatases. In the phosphatase reaction, the enzyme bound nucleophile can be serine, lysine, or cysteine. The 3′-phosphodiesterase activity and tyrosine phosphatases do have similar substrates: phosphodiester vs. phosphate linked to tyrosine. Therefore, the phosphodiesterase activity does not result from a simple hydrolysis reaction. Mechanism based inhibitors can be developed because both potential mechanisms rely upon an enzyme-bound hydrolysis step.

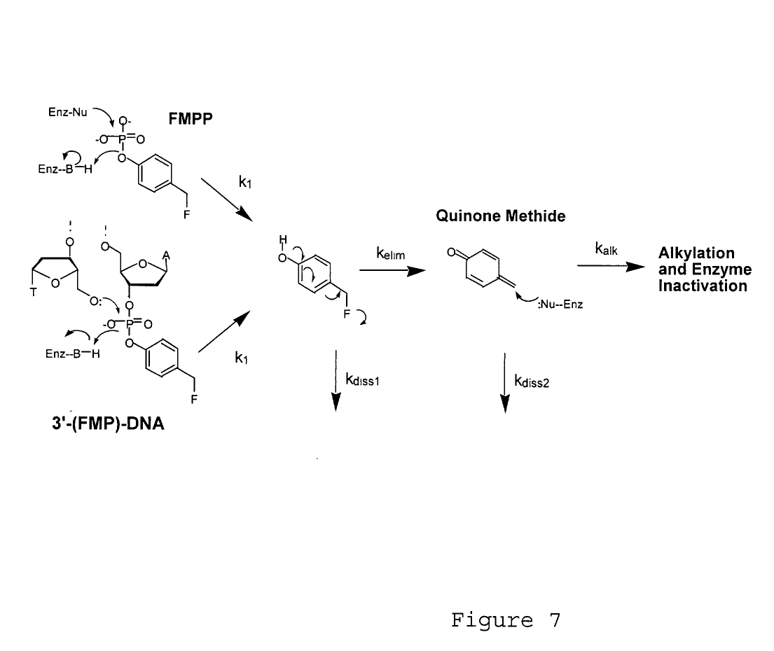

The compound 4-(fluoromethyl)phenyl phosphate (FMPP) has been demonstrated to be a potent inhibitor of prostatic acid phosphatase (PAP)(Myers, J., and Widlanski, T., Science 262:1451-1453 (1993)). In this reaction, PAP catalyzes the hydrolysis of the phosphate ester bond (rate constant k1 in FIG. 7) and leads to the formation of metastable phenoxide at the active site. This phenoxide eliminates a fluoride ion (keₑₗᵢₘ) to give a quinone methide, a very powerful alkylating agent, and can inactivate the enzyme through alkylation (kₐₗₖ) of an active site residue. Similar substrates have been used to study RNAse A (Stowell et al., J. Org. Chem. 60:6930-6936 (1995)) and calcinurin (Born et al., J. Biol. Chem. 270:25651-25655 (1995)).

It follows that DNA containing 3′-(4-(fluoromethyl)phenyl)-phosphate (3′-[FMPP]-DNA) can specifically alkylate TDP. The nucleophilic displacement of tyrosine (k₁) by water or some other nucleophile is analogous to the nucleophilic displacement of tyrosine during the phosphatase reaction; both are phosphoryl transfer reactions (SN2 nucleophilic displacement reactions) and in both cases the leaving group is tyrosine. The critical feature of this approach is the generation of a quinone methide at the active site of the enzyme and is therefore independent of the attacking nucleophile.

A feature of this approach is that the rate of fluoride elimination (kₑₗᵢₘ) must be faster than the rate of dissociation (k_{diss1}); and the rate of alkylation (kₐₗₖ) must be faster than the rates of dissociation (k_{diss2}). This can only be determined experimentally. If the rate of elimination is too slow relative to dissociation (kdiss1), the reagent can be modified to increase this rate. For example, if the tyrosine bridging oxygen is replaced with sulfur, the rate of elimination will increase because the sulfhydryl will deprotonate faster leading to a faster formation of the quinone methide. This,modification should also increase the rate of cleavage (k₁) because sulfur-phosphorous bonds are less stable than oxygen-phosphorous bonds. The rate of alkylation is not easily manipulated because it will most likely be influenced by the positioning of an enzyme bound nucleophile (:Nu-Enz in FIG. 7). 3′-(FMP)-nucleotides can be synthesized, for example, as described in Stowell et al., J. Orq. Chem. 60:6930-6936 (1995). 3′-(FMP)-oligonucleotides can be synthesized using similar methods.

### EXAMPLE VI

### Small Molecule Library Screening

This example describes the use of an exemplary small molecule library for screening to identify compounds that modulate TDP.

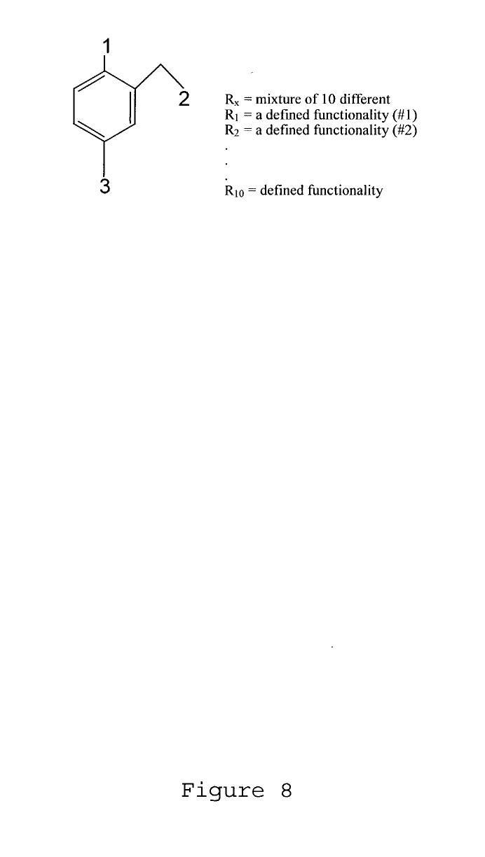

A small molecule library, such as the library shown in FIG. 8, can be screened using the methods described herein to identify TDP modulating compounds. In the compound library depicted in FIG. 8, a single phenyl ring can be derivatized with many different functional groups at three different positions (1, 2 or 3). If 10 different functional groups are placed at each of these positions, then 1000 different compounds will be present within the complete library. Such a library can have few compounds or several thousand compounds, depending on the desired properties of the compounds to be screened.

Two general approaches have been described for the identification of individual active compounds from synthetic libraries: iterative deconvolution vs. positional scanning (Ostresh,et al. Methods Enzymol. 267, 220-234 (1996)). Because the current TDP assay is sensitive, the positional scanning method can be used to deconvolute the combinatorial libraries. In this approach, sublibraries are constructed based on the number of different susbstituted positions. As shown in FIG. 9, three different sublibraries can be constructed to deconvolute the library represented in FIG. 8. Each sublibrary can be analyzed by synthesizing molecules with a defined functionality (R1-R10) at one position and a mixture of functional groups (Rx) at each of the other two positions. For example, sublibrary 1 can be analyzed by determining which functional group results in the most inhibition of TDP when position 2 and 3 are mixtures of all possible combinations. If 10 different functionalities are possible, then 10 different assays can be run in order to define which functionality results in the greatest inhibition. In other words, which functionality results in the greatest enzyme inhibition at position 1 when positions 2 and 3 are complete mixtures. As an illustration, the example in FIG. 9 defines functionality R5 to be most potent at position 1. The same analysis is then repeated at positions 2 and 3. In the example, functionality R7 is ideal at position 2 and R9 is ideal at position 3. From these results it is possible to identify the idealized inhibitor. In this example, the best inhibitor can be identified from a mixture of 1000 different compounds by simply performing 30 different assays.

A step in this process is identifying the best functionality at each diversity position. This can be accomplished by not only determining the amount enzyme inhibition, but the amount of inhibition as a function of inhibitor concentration (mixture concentration). The mixture that results in the greatest inhibition at the lowest concentration (IC50) will identify the best functionality at a given position.

Throughout this application various publications have been referenced within parentheses. The disclosures of these publications in their entireties are hereby incorporated by reference in this application in order to more fully describe the state of the art to which this invention pertains.

Although the invention has been described with reference to the disclosed embodiments, those skilled in the art will readily appreciate that the specific experiments detailed are only illustrative of the invention. It should be understood that various modifications can be made without departing from the spirit of the invention. Accordingly, the invention is limited only by the following claims.

What is claimed is:

1. A compound of the formula: 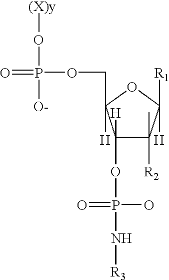
wherein,
X is a nucleotide;
y is a positive integer;
R1 is any nucleotide base;
R2 is H; and
R3 is phenyl or substituted phenyl.

2. The compound of claim 1, wherein R2 is H.

3. The compound of claim 2, wherein R3 is phenyl.

4. A composition, comprising the compound of claim 1 and a camptothecin.

5. A method of inhibiting 3′-tyrosyl-DNA phophodiesterase (TDP) activity, comprising contacting a TDP with a compound of claim 1.

6. A method of decreasing cellular proliferation, comprising contacting a TDP-containing cell with an effective amount of a TDP inhibiting compound sufficient to inhibit TDP activity in said cell.

7. The method of claim 6, wherein said TDP inhibiting compound is selected from the group consisting of polynucleotide-3′-bridging phosphoramidate, polynucleotide-3′-alkyl phosphonate, polynucleotide-3′-alkyl phosphotriester, polynucleotide-bridging-alkyl phosphonate, nucleotide-3′-bridging phosphoramidate, nucleotide-3′-alkyl phosphonate, nucleotide-3′-alkyl phosphotriester, and nucleotide-bridging-alkyl phosphonate.

8. The method of claim 6, wherein said cell is a cancer cell.

9. The method of claim 8, wherein said cancer cell is a cancer cell selected from the group consisting of ovarian, small-cell, breast, leukemia and lymphoma cancer cell.

10. A method of reducing the severity of a proliferative disease, comprising administering to an individual an effective amount of a TDP inhibiting compound sufficient to inhibit the activity of TDP in a cell in said individual, thereby decreasing cell proliferation to reduce the severity of said proliferative disease.

11. The method of claim 10, wherein said TDP inhibiting compound is selected from the group consisting of polynucleotide-3′-bridging phosphoramidate, polynucleotide-3′-alkyl phosphonate, polynucleotide-3′-alkyl phosphotriester, polynucleotide-bridging-alkyl phosphonate, nucleotide-3′-bridging phosphoramidate, nucleotide-3′-alkyl phosphonate, nucleotide-3′-alkyl phosphotriester, and nucleotide-bridging-alkyl phosphonate.

12. The method of claim 10, wherein said proliferative disease is a cancer selected from the group consisting of ovarian cancer, small-cell cancer, breast cancer, leukemia and lymphoma.

13. A method of screening for compounds that modulate the activity of TDP, comprising:
(a) contacting TDP, or a fragment or modification thereof having TDP activity, with a compound under conditions that allow TDP activity;
(b) determining an amount of TDP activity; and
(c) identifying a compound that modulates TDP activity.
